SEQUENCE LISTING (62 sequences)

SEQ ID NO: 1 - DNA, 23 bp, Artificial Sequence
  Primer
ccccagggccccattttggtacc

SEQ ID NO: 2 - DNA, 20 bp, Artificial Sequence
  Primer
ggcacaaactccaggttttc

SEQ ID NO: 3 - DNA, 20 bp, Artificial Sequence
  Primer
acactgcccctctcacacat

SEQ ID NO: 4 - DNA, 22 bp, Artificial Sequence
  Primer
gggttttctccatgctgtttct

SEQ ID NO: 5 - DNA, 22 bp, Artificial Sequence
  Primer
acccacacaggtgagaaacctt

SEQ ID NO: 6 - DNA, 22 bp, Artificial Sequence
  Primer
gttgggaacttgaccatgattg

SEQ ID NO: 7 - DNA, 22 bp, Artificial Sequence
  Primer
agcagaggagcaaaagctcatt

SEQ ID NO: 8 - DNA, 22 bp, Artificial Sequence
  Primer
ccaaagtccaatttgaggcagt

SEQ ID NO: 9 - DNA, 23 bp, Artificial Sequence
  Primer
ccccagggccccattttggtacc

SEQ ID NO: 10 - DNA, 22 bp, Artificial Sequence
  Primer
aacctacaggtggggtctttca

SEQ ID NO: 11 - DNA, 20 bp, Artificial Sequence
  Primer
acactgcccctctcacacat

SEQ ID NO: 12 - DNA, 22 bp, Artificial Sequence
  Primer
aacctacaggtggggtctttca

SEQ ID NO: 13 - DNA, 20 bp, Artificial Sequence
  Primer
gaccacctcgccttacacat

SEQ ID NO: 14 - DNA, 22 bp, Artificial Sequence
  Primer
aacctacaggtggggtctttca

SEQ ID NO: 15 - DNA, 22 bp, Artificial Sequence
  Primer
agcagaggagcaaaagctcatt

SEQ ID NO: 16 - DNA, 22 bp, Artificial Sequence
  Primer
aacctacaggtggggtctttca

SEQ ID NO: 17 - DNA, 19 bp, Artificial Sequence
  Primer
tagctgtgctcgggctact

SEQ ID NO: 18 - DNA, 18 bp, Artificial Sequence
  Primer
tctctgctggatgacgcg

SEQ ID NO: 19 - DNA, 19 bp, Artificial Sequence
  Primer
acatcgctcagacaccatg

SEQ ID NO: 20 - DNA, 22 bp, Artificial Sequence
  Primer
tgtagttgaggtcaatgaaggg

SEQ ID NO: 21 - DNA, 21 bp, Artificial Sequence
  Primer
aggagatttggctgagattgg

SEQ ID NO: 22 - DNA, 22 bp, Artificial Sequence
  Primer
tttcctacatgagcatccactg

SEQ ID NO: 23 - DNA, 17 bp, Artificial Sequence
  Primer
gacccgtcaaacccagg

SEQ ID NO: 24 - DNA, 19 bp, Artificial Sequence
  Primer
gaggctgacccataacagg

SEQ ID NO: 25 - DNA, 19 bp, Artificial Sequence
  Primer
acggcagatcatgtttccc

SEQ ID NO: 26 - DNA, 23 bp, Artificial Sequence
  Primer
cacaagcattaaacagtggagtg

SEQ ID NO: 27 - DNA, 20 bp, Artificial Sequence
  Primer
tctttggaatgcatggaggg

SEQ ID NO: 28 - DNA, 21 bp, Artificial Sequence
  Primer
gtagatgaaagccagacccag

SEQ ID NO: 29 - DNA, 23 bp, Artificial Sequence
  Primer
tcttcataacactgacctggaac

SEQ ID NO: 30 - DNA, 22 bp, Artificial Sequence
  Primer
accatccttttcgtcatcttcc

SEQ ID NO: 31 - DNA, 21 bp, Artificial Sequence
  Primer
ccctctttgtacatgagcact

SEQ ID NO: 32 - DNA, 22 bp, Artificial Sequence
  Primer
acttgatgtcttccactagctg

SEQ ID NO: 33 - DNA, 19 bp, Artificial Sequence
  Primer
aaccagtgccagcatactc

SEQ ID NO: 34 - DNA, 21 bp, Artificial Sequence
  Primer
ctcgtgaatctcctgcttcag

SEQ ID NO: 35 - DNA, 21 bp, Artificial Sequence
  Primer
caaacaagaggtccctggatc

SEQ ID NO: 36 - DNA, 20 bp, Artificial Sequence
  Primer
agttgatgcctggtctgaag

SEQ ID NO: 37 - DNA, 20 bp, Artificial Sequence
  Primer
gatcaggtctgggtgctaag

SEQ ID NO: 38 - DNA, 22 bp, Artificial Sequence
  Primer
ccactctgtcaaattgctttcc

SEQ ID NO: 39 - DNA, 18 bp, Artificial Sequence
  Primer
ggctcctgcaaatgcaaa

SEQ ID NO: 40 - DNA, 18 bp, Artificial Sequence
  Primer
cagcagctgcacttgtcc

SEQ ID NO: 41 - DNA, 20 bp, Artificial Sequence
  Primer
gcgtcacgctccgagccttt

SEQ ID NO: 42 - DNA, 20 bp, Artificial Sequence
  Primer
ctgagcatcgcagccgcagt

SEQ ID NO: 43 - DNA, 24 bp, Artificial Sequence
  Primer
cttggtgctttggaactgcgacat

SEQ ID NO: 44 - DNA, 24 bp, Artificial Sequence
  Primer
ttgcacagtggtttcctcaaagcc

SEQ ID NO: 45 - DNA, 20 bp, Artificial Sequence
  Primer
ctggctgagggcttctacac

SEQ ID NO: 46 - DNA, 20 bp, Artificial Sequence
  Primer
cctagccattttcccaaaca

SEQ ID NO: 47 - DNA, 19 bp, Artificial Sequence
  Primer
acaggcttccattgaccag

SEQ ID NO: 48 - DNA, 21 bp, Artificial Sequence
  Primer
tcaccatagagtgcttccaac

SEQ ID NO: 49 - DNA, 19 bp, Artificial Sequence
  Primer
agaccctacatcctcaccc

SEQ ID NO: 50 - DNA, 23 bp, Artificial Sequence
  Primer
aaagagaacgacacctatcatgg

SEQ ID NO: 51 - DNA, 21 bp, Artificial Sequence
  Primer
tggttcaggccattaactcag

SEQ ID NO: 52 - DNA, 20 bp, Artificial Sequence
  Primer
tctcccgcttgttaatgtcg

SEQ ID NO: 53 - DNA, 19 bp, Artificial Sequence
  Primer
agttccacaagctaggcac

SEQ ID NO: 54 - DNA, 21 bp, Artificial Sequence
  Primer
acttatagcggcaatcgtcag

SEQ ID NO: 55 - DNA, 20 bp, Artificial Sequence
  Primer
tgcaactggagaggatcaac

SEQ ID NO: 56 - DNA, 20 bp, Artificial Sequence
  Primer
ccgaaagatctgaccgaagg

SEQ ID NO: 57 - DNA, 23 bp, Artificial Sequence
  Primer
cagtttccaaaccagatgtcatc

SEQ ID NO: 58 - DNA, 21 bp, Artificial Sequence
  Primer
actgggagttgtgaaggttac

SEQ ID NO: 59 - DNA, 20 bp, Artificial Sequence
  Primer
gagaaggagaagctggagca

SEQ ID NO: 60 - DNA, 20 bp, Artificial Sequence
  Primer
gtgaagtgagggctcccata

SEQ ID NO: 61 - DNA, 20 bp, Artificial Sequence
  Primer
acctggcgagtctgacatgg

SEQ ID NO: 62 - DNA, 23 bp, Artificial Sequence
  Primer
tcttcatgtgtaaggcgaggtgg

(19) United States
(12) Patent Application Publication
(10) Pub. No.: US 2016/0230227 A1
(43) Pub. Date: Aug. 11, 2016

(54) METHODS OF TREATING ALZHEIMER'S DISEASE

(71) Applicant: THE NEW YORK STEM CELL FOUNDATION, New York, NY (US)

(72) Inventors: Andrew Sproul, New York, NY (US); Samson Jacob, Carmel, NY (US); Scott Noggle, New York, NY (US)

(21) Appl. No.: 14/653,832

(22) PCT Filed: Dec. 20, 2013
(86) PCT No.: PCT/US2013/077259

§ 371 Date: Jun. 18, 2015

Related U.S. Application Data
(60) Provisional application No. 61/745,356, filed on Dec. 21, 2012.

(51) Int. Cl. C12Q 1/68; A61K 31/473; G06F 19/20; A61K 31/445; A61K 31/13; A61K 31/55; A61K 31/325

(52) U.S. Cl. CPC C12Q 1/6883; A61K 31/55; A61K 31/473; A61K 31/325; A61K 31/445; A61K 31/13; G06F 19/20; C12Q 2600/158; C12Q 2600/16; C12Q 2600/118; C12Q 2600/136; C12Q 2600/106

(57) ABSTRACT

The present invention relates to the diagnosis, prognosis, progression, and treatment of Alzheimer's disease. In particular, Alzheimer's disease can be characterized by the differential expression of certain genes, including ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1. The present invention also relates to systems and methods of analyzing Alzheimer's disease, methods of screening for treatment agents for Alzheimer's disease and kits for the analysis of Alzheimer's disease.

### CROSS REFERENCE TO RELATED APPLICATIONS

This application claims the benefit of priority under §119(e) of U.S. Ser. No. 61/745,356, filed Dec. 21, 2012. The disclosure of the prior application is considered part of and is incorporated by reference in its entirety in the disclosure of this application.

### GRANT INFORMATION

This invention was made with government support under Grant Nos. R21AG042965, NS049442, and R01-MH091844 awarded by the National Institutes of Health. The government has certain rights in the invention.

### INCORPORATION OF SEQUENCE LISTING

The material in the accompanying Sequence Listing is hereby incorporated by reference into this application. The accompanying sequence listing text file, name NYSC1160_1WO_Sequence_Listing, was created on Dec. 20, 2013 and is 12 KB. The file can be assessed using Microsoft Word on a computer that uses Windows OS

### BACKGROUND OF THE INVENTION

1. Field of the Invention

The invention relates generally to the diagnosis, prognosis, progression, and treatment of Alzheimer's disease and more specifically to the differential expression profile characteristic of Alzheimer's disease.

2. Background Information

Although the majority of Alzheimer's disease (AD) cases are late onset and likely result from a mixture of genetic predisposition and environmental factors, there are autosomal dominant genetic forms of the disease that affect patients at much earlier ages (FAD). Known familial early-onset genes include mutations in amyloid precursor protein (APP), presenilin-1 (PSEN/PS1), and presenilin-2 (PSEN2/PS2). PSEN1 mutations are responsible for the most common form of inherited AD. The most prevalent theory for the underlying cause of AD is the “amyloid hypothesis”, in which toxic oligomerogenic forms of Aβ, a cleavage product of APP, accumulate and cause neuronal dysfunction and cell death. PS1/PS2 are key components of the γ-secretase complex that mediates one of the two APP cleavage events, and mutations in PS1 increase the relative ratios of the more oligomerogenic Aβ species (i.e. Aβ42) to less oligomerogenic species (Aβ40).

Most investigation of the molecular phenotypes caused by the PSEN1 mutations has focused on this microheterogeneous cleavage at the carboxy terminus of Aβ. This qualitative change is believed to be associated with hypomorphism in processivity and has implications for misprocessing of multiple substrates other than APP. Further, the magnitude of the mutant PSEN1-associated perturbations of Aβ42/Aβ40 varies widely, and, in some mutations (e.g., PSEN1 L271V in the Tas-1 family) alterations in the Aβ42/Aβ40 ratio have been either minimal or difficult to demonstrate. This raises the possibility that PS1 could have physiological or pathological effects independent of its effects on APP processing. This is an important issue to investigate thoroughly since PSEN1 mutations are present in virtually all of the cell- and mouse-based models used to develop hypotheses and treatments for common, sporadic AD. However, in common, sporadic AD, no PSEN1 mutation is present. Indeed, PSEN1-mutation-related AD is conceived as a disease of Aβ anabolism while at least some forms of common, sporadic AD (i.e., that linked to APOE ε4) are conceived as a disease of Aβ catabolism. Other genes linked to common, sporadic AD (e.g., CR1) appear to act via the immune response and may modulate cerebral amyloidosis in unexpected ways.

Recently several groups have generated human iPSC or transdifferentiation models of AD, with studies primarily focused on FAD neurons. None of these studies addressed whether there are any differences between AD and control NPCs prior to neuronal differentiation. NPCs are a potentially relevant system to study aspects of disease on neuronal differentiation. Some FAD mouse models demonstrate deficits in neurogenesis as the animals age, and NPCs taken from AD brains of recently deceased patients have decreased neurogenic potential in comparison to those from similarly aged healthy controls. Newly born adult neurons in mouse models of AD have also been reported to have significantly decreased viability relative to control mice. In addition, the brains of early-onset Alzheimer's patients might have developmental alterations that could affect the progression of the disease. This possibility has been recently speculated in response to a report that young adults from the Colombian FAD kindred (PS1 E280A) have changes in grey matter and synaptic function potentially prior to formation of Aβ plaques. NPCs are also a more homogenous population that might reduce the experimental variability of mature neurons produced by current neuronal differentiation protocols, and thus could be a better system to identify novel molecules potentially important for early events in AD.

### SUMMARY OF THE INVENTION

The present invention relates to the diagnosis, prognosis, progression, and treatment of Alzheimer's disease. In particular, the present invention is based on the discovery that Alzheimer's disease can be characterized by the differential expression of certain genes, including at least one of ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1. The present invention also relates to systems and methods of analyzing Alzheimer's disease, methods of screening for treatment agents for Alzheimer's disease and kits for the analysis of Alzheimer's disease.

Disclosed herein is a method of diagnosing, prognosing, determining progression of Alzheimer's disease, predicting a therapeutic regimen or predicting benefit from therapy in a subject having Alzheimer's disease comprising assaying an expression level of at least one or more of a plurality of targets in a sample from the subject, wherein the plurality of targets comprises one or more targets selected from NLRP2, ASB9, BIK, C7orfl6, ZNF300 or ADM2 or any combination thereof; and diagnosing, prognosing, determining progression AD, predicting a therapeutic regimen or predicting benefit from therapy in a subject having AD based on the expression levels of the plurality of targets. In one embodiment, the plurality of targets further comprises targets selected from one or more of NDP, PLP1, SLC45A2, TBX2, TUBB4, FLJ35024, MT2A, PTGS2, ABCC2, ECEL1, EGFL8, FSTL5, or SMOC1 or any combination thereof. In an additional embodiment, ASB9, BIK, C7orfl6, NDP, NLRP2, PLP1, SLC45A2, TBX2, TUBB4 and/or ZNF300 are upregulated. In another embodiment, ADM2, FLJ35024, MT2A and/or PTGS2 are downregulated. In some embodiments, the plurality of targets comprises two or more targets. In some embodiments, the plurality of targets comprises 2, 3, 4, 5, 6, 7, 8, 9, 10, 11, 12, 13, 14, 15, 16, 17, 18 or 19 or more targets. In some embodiments, the method further comprises amplifying the one or more targets, conducting a multiplexed reaction, sequencing the plurality of targets, quantifying the plurality of targets, hybridizing a label to a plurality of targets, amplifying the plurality of targets, reverse transcribing the plurality of targets, or hybridizing a probe to the plurality of targets, In some embodiments, the probe or plurality of targets is labeled. In some embodiments, the method further comprises attaching the plurality of targets to a solid support. In some embodiments, the method further comprises attaching a probe or probe set to a solid support. In some embodiments, the solid support is an array, a bead, a plate, or microwell plate.

Also disclosed herein is a system for analyzing Alzheimer's disease in a subject, comprising a probe set comprising a plurality of target sequences, wherein the plurality of target sequences hybridizes to one or more targets which may be NLRP2, ASB9, BIK, C7orfl6, ZNF300 and/or ADM2 or any combination thereof; a plurality of probes that hybridize to the plurality of target sequences; a sample from a subject having Alzheimer's disease; a computer model or algorithm for analyzing an expression level and/or expression profile of the targets hybridized to the probes in a sample from a subject having Alzheimer's disease. In some embodiments, the system further comprises an electronic memory for capturing and storing an expression profile; a computer-processing device, optionally connected to a computer network; a software module executed by the computer-processing device to analyze an expression profile; a software module executed by the computer-processing device to compare the expression profile to a standard or control; a software module executed by the computer-processing device to determine the expression level of the target; a machine to isolate the target or the probe from the sample; a machine to sequence the target or the probe; a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises two or more targets. In some embodiments, the plurality of targets comprises 2, 3, 4, 5, 6, 7, 8, 9, 10, 11, 12, 13, 14, 15, 16, 17, 18 or 19 or more targets. In additional embodiments, the target further comprises NDP, PLP1, SLC45A2, TBX2, TUBB4, FLJ35024, MT2A, PTGS2, ABCC2, ECEL1, EGFL8, FSTL5, or SMOC1 or any combination thereof.

Further disclosed herein is a method of treating a subject with Alzheimer's disease comprising obtaining an expression profile of a plurality of targets from a sample from a subject; determining the subject has Alzheimer's disease by comparing the expression profile from the patient to an expression profile from a control or standard; and administering treatment to the subject, wherein the plurality of targets comprises one or more of the following targets NLRP2, ASB9, BIK, C7orfl6, ZNF300 and ADM2 or any combination thereof. In an embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In some embodiments, ASB9, BIK, C7orfl6, NDP, NLRP2, PLP1, SLC45A2, TBX2, TUBB4 and/or ZNF300 are upregulated. In additional embodiments, ADM2, FLJ35024, MT2A and/or PTGS2 are downregulated. In further embodiments, the treatment is an acetylcholinesterase inhibitor or a an NMDA receptor antagonist. In another embodiment, the treatment maybe tacrine, rivastigmine, galantamine, donepezil or memantine.

Additionally disclosed herein is a method of identifying an agent for the treatment of Alzheimer's disease comprising: contacting a cell with a test agent, wherein the expression of a plurality of targets in the cell is consistent with Alzheimer's disease; determining the expression of the plurality of targets from the cell; comparing the expression of the plurality of targets from the cell to a control or standard; and determining that the expression of the plurality of targets is not consistent with Alzheimer's disease, wherein the plurality of targets comprises one or more of the following targets NLRP2, ASB9, BIK, C7orfl6, ZNF300 and ADM2 or any combination thereof. In an embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In one embodiment, up regulation of ASB9, BIK, C7orfl6, NDP, NLRP2, PLP1, SLC45A2, TBX2, TUBB4 and/or ZNF300 is consistent with Alzheimer's disease. In a further embodiment, downregulation of ADM2, FLJ35024, MT2A and/or PTGS2 is consistent with Alzheimer's disease.

Further disclosed herein is a kit for analyzing Alzheimer's disease, comprising: a probe set comprising a plurality of target sequences, wherein the plurality of target sequences hybridizes to more than one target of the following NLRP2, ASB9, BIK, C7orfl6, ZNF300 and ADM2 or any combination thereof; and a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. altering a treatment regimen. In a further embodiment, the target further comprises NDP, PLP1, SLC45A2, TBX2, TUBB4, FLJ35024, MT2A, PTGS2, ABCC2, ECEL1, EGFL8, FSTL5, or SMOC1 or a combination thereof.

Also disclosed herein is a system for analyzing Alzheimer's disease comprising: a computer processing device for determining an expression profile for a plurality of targets; an electronic memory device for capturing and storing an expression profile; a software module; a machine to isolate target or the probe from the sample; a machine to sequence the target or the probe; a machine to amplify the target or the probe; a computer model or algorithm for analyzing an expression level and/or expression profile of the target hybridized to the probe in a sample from a subject suffering from AD; and an output providing the analysis. In one embodiment, the a plurality of targets comprises one or more of the following targets NLRP2, ASB9, BIK, C7orfl6, ZNF300 and ADM2 or any combination thereof. In another embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or a combination thereof. In an embodiment, the software module executed by the computer-processing device analyzes an expression profile. In some embodiments, the software compare executed by the computer-processing devices the expression profile to a standard or control; determines the expression level of the target; transmits an analysis of the expression profile to the subject or a medical professional treating the subject. In another embodiment, the system further comprises a label that specifically binds to the target, the probe, or a combination thereof.

Additionally disclosed herein is a method for analyzing Alzheimer's disease comprising a computer processing device for determining an expression profile for a plurality of targets; an electronic memory device for capturing and storing an expression profile; a software module; a machine to isolate target or the probe from the sample; a machine to sequence the target or the probe; a machine to amplify the target or the probe; a computer model or algorithm for analyzing an expression level and/or expression profile of the target hybridized to the probe in a sample from a subject suffering from AD; and an output providing the analysis. In one embodiment, the plurality of targets comprises one or more of the following targets NLRP2, ASB9, BIK, C7orfl6, ZNF300 and ADM2 or any combination thereof. In another embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof or a combination thereof. In an embodiment, the software module executed by the computer-processing device analyzes an expression profile. In some embodiments, the software compare executed by the computer-processing devices the expression profile to a standard or control; determines the expression level of the target; transmits an analysis of the expression profile to the subject or a medical professional treating the subject. In another embodiment, the method further comprises a label that specifically binds to the target, the probe, or a combination thereof.

Disclosed herein is a method of diagnosing, prognosing, determining progression of Alzheimer's disease, predicting a therapeutic regimen or predicting benefit from therapy in a subject having Alzheimer's disease comprising assaying an expression level a protein encoded by at least one or more of a plurality of targets in a sample from the subject, wherein the plurality of targets comprises one or more targets selected from NLRP2, ASB9, BIK, C7orfl6, ZNF300 or ADM2 or any combination thereof; and diagnosing, prognosing, determining progression AD, predicting a therapeutic regimen or predicting benefit from therapy in a subject having AD based on the protein expression levels of the plurality of targets. In one embodiment, the plurality of targets further comprises targets selected from one or more of NDP, PLP1, SLC45A2, TBX2, TUBB4, FLJ35024, MT2A, PTGS2, ABCC2, ECEL1, EGFL8, FSTL5, or SMOC1 or any combination thereof. In an additional embodiment, ASB9, BIK, C7orfl6, NDP, NLRP2, PLP1, SLC45A2, TBX2, TUBB4 and/or ZNF300 are upregulated. In another embodiment, ADM2, FLJ35024, MT2A and/or PTGS2 are downregulated. In some embodiments, the plurality of targets comprises two or more targets. The protein expression level may be determined by routine laboratory methods. These methods include, but are not limited to, ELISA, RIA and FACs analysis.

### BRIEF DESCRIPTION OF THE DRAWINGS

FIGS. 1A-F show iPSC characterization and neuronal differentiation. A. Sequencing of core set iPSCs for PSEN1 mutations in exon 5 (M146L) and exon 8 (A246E) respectively. * marks site of the mutations. B. Cartoon of neuronal differentiation scheme, showing timing and changes into different medias. C. All 8 core lines were neuronally differentiated for 14 days and were analyzed by flow cytometry for the expression of CD56 (NCAM). D. Representative immunostaining for the neural progenitor marker nestin in iPSC line 8446B. E. Quantification of nestin staining F. Representative immunostaining for the cell cycle marker Ki67 in cell line 7889O. G. Quantification of ki67.

FIGS. 2A-E show the action potentials and normal spontaneous Ca²⁺ transients are present in neurons differentiated from control line 7889O and 8446. A. Representative traces in voltage clamp mode showing fast inward currents followed by long-lasting outward currents. Voltage 10 mV steps are shown in the upper panel. The inset shows an enlarged view of the inward current. (7899O, day 55). B. Representative action potentials in response to step current injections of 20 pA (lower panel) in current clamp mode (7889O, day 45). C. Representative action potentials in response to step current injections of 20 pA as in B in current clamp mode (7889O, day 55). D. Representative action potentials in response to step current injections of 20 pA from 8446B, day 55. E. (A) Image of 7889O loaded with Fluo-4NW and stained for MAP2 after Ca²⁺ transients have been recorded. (B) Representative spontaneous Ca²⁺ spikes recorded from 7889O neurons before and after application of TTX.

FIGS. 3A-B show that Aβ42/Aβ40 ratio is elevated in PSEN1 cells. A. Aβ42/Aβ40 ratio is increased in day 14 differentiated NPCs/early neurons. B. Aβ 42/40 ratios are shown for both control and PSEN1 fibroblasts and NPCs. C. Total Aβ levels (Aβ40+Aβ42) are statistically similar between control and PSEN1 fibroblasts and NPCs/early neurons.

FIGS. 4A-C show gene expression profiling of control vs. PS1 NPCs/early neurons. A. Clustering of 8 core lines by correlation. UR stands for unrelated control. B. Scatter plot (log scale) of the correlation of gene expression between 4 control lines and 4 PSEN1 lines. C. Chart indicating the number of upregulated and downregulated genes for each threshold of analysis.

FIGS. 5A-D show validation of target genes in PSEN1 NPCs. A-B. NLRP2 mRNA expression was assessed in undifferentiated iPSCs. C. Western blot analysis of NLRP2 protein expression in NPCs. D. Representative experiment showing ASB9 mRNA expression in NPCs.

FIGS. 6A-C show examination of target genes in late-onset AD brains. A-B. qPCR of NLRP2 (A) and ASB9 (B) from mRNA from Brodmann's area (BA38) from control and AD brains. C. List of PSEN1 NPC target genes (Table 2) that have differential expression in independent microarray data of laser captured microdissected (LCM) cortical neurons from one of three brain areas (details in FIG. 11).

FIGS. 7A-E show additional characterization of 7768C and neuronal differentiation. A. Real-time (RT) PCR of viral-specific transgene markers using RNA from virally infected 293 cells as a positive control. B. RT-PCR of endogenous stem cell genes, using RNA from human embryonic stem cells as a positive control, and virally infected HEK293 as a negative control. C. 7768C demonstrates a normal female karyotype as measured by G-banding. D. In vivo pluripotency was assessed by injection of undifferentiated 7768C iPSCs into NSG mice and harvesting the resulting teratoma for analysis. E. Day 14 differentiated cells produced patches of Tuj1+ neurons with complex morphology.

FIGS. 8A-H show characterization of core lines. A. Alkaline-Phosphatase activity assay. B-C. Immunofluorescence for relevant stem cell markers. B. SSEA4, Oct4. C. Tra160. D-F. Immunofluorescence for three germ layers formed by Embryoid Bodies (EBs): D, Ectoderm, E, Mesoderm, F, Endoderm. G-H. Southern blotting using probes for either OCT4 or KLF4.

FIG. 9 shows DapT blocks total AI production.

FIGS. 10A-E show NDP protein is expressed in Late-Onset AD Brains. A. Description of samples utilized for FIG. 6A,B and panel B. B. qPCR of NDP expression in area BA38 of control and AD patients as in FIG. 6. C-E. Expression of NDP in a Late-Onset AD Brain, showing NDP DAB reactivity in neuronal cytoplasm and axons (C), neuronal nuclei (D) and some neurofibrillary tangles (E).

FIGS. 11A-B shows recombinant Norrin protein induces proliferation in adult SVZ neural progenitor cells (NPCs). A. The number of neurospheres formed after 10 days in vitro. B. Representative images of neurosphere formation assay showing the effect of recombinant Norrin protein (100 ng/ml) on the treated NPCs. Error bars represent standard error of the mean. *D<0.05, analysis of variance with Dunnet's post hoc analysis.

### DETAILED DESCRIPTION OF THE INVENTION

The present invention is based on the discovery that Alzheimer's disease (AD) can be characterized by differentially expressed genes. The present invention discloses systems and methods for diagnosing, predicting, and/or monitoring the status or outcome of AD in a subject using expression-based analysis differentially expressed genes.

Generally, the method comprises (a) providing a sample from a subject suffering from AD; (b) assaying the expression level for a plurality of targets in the sample; and (c) diagnosing, predicting and/or monitoring the status or outcome of AD based on the expression level of the plurality of targets. The present invention also discloses methods of treating AD, methods for screening agents to treat AD and kits for the analysis of AD.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Assaying the expression level for a plurality of targets in the sample may comprise applying the sample to a microarray. In some instances, assaying the expression level may comprise the use of an algorithm. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

The term “polynucleotide” as used herein refers to a polymer of greater than one nucleotide in length of ribonucleic acid (RNA), deoxyribonucleic acid (DNA), hybrid RNA/DNA, modified RNA or DNA, or RNA or DNA mimetics, including peptide nucleic acids (PNAs). The polynucleotides may be single- or double-stranded. The term includes polynucleotides composed of naturally-occurring nucleobases, sugars and covalent internucleoside (backbone) linkages as well as polynucleotides having non-naturally-occurring portions which function similarly. Such modified or substituted polynucleotides are well known in the art and for the purposes of the present invention, are referred to as “analogues.” One of skill in the art will readily understand the sequences for polynucleotides may be designated by a GenBank accession number and that the sequences are publically available and can be obtained using the GenBank accession number at www.ncbi.nlm.nih.gov.

“Complementary” or “substantially complementary” refers to the ability to hybridize or base pair between nucleotides or nucleic acids, such as, for instance, between a sensor peptide nucleic acid or polynucleotide and a target polynucleotide. Complementary nucleotides are, generally, A and T (or A and U), or C and G. Two single-stranded polynucleotides or PNAs are said to be substantially complementary when the bases of one strand, optimally aligned and compared and with appropriate insertions or deletions, pair with at least about 80% of the bases of the other strand, usually at least about 90% to 95%, and more preferably from about 98 to 100%.

Alternatively, substantial complementarity exists when a polynucleotide may hybridize under selective hybridization conditions to its complement. Typically, selective hybridization may occur when there is at least about 65% complementarity over a stretch of at least 14 to 25 bases, for example at least about 75%, or at least about 90% complementarity.

“Preferential binding” or “preferential hybridization” refers to the increased propensity of one polynucleotide to bind to its complement in a sample as compared to a noncomplementary polymer in the sample.

Hybridization conditions may typically include salt concentrations of less than about 1M, more usually less than about 500 mM, for example less than about 200 mM. In the case of hybridization between a peptide nucleic acid and a polynucleotide, the hybridization can be done in solutions containing little or no salt. Hybridization temperatures can be as low as 5° C., but are typically greater than 22° C., and more typically greater than about 30° C., for example in excess of about 37° C. Longer fragments may require higher hybridization temperatures for specific hybridization as is known in the art. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art.

“Multiplexing” herein refers to an assay or other analytical method in which multiple analytes are assayed. In some instances, the multiple analytes are from the same sample. In some instances, the multiple analytes are assayed simultaneously. Alternatively, the multiple analytes are assayed sequentially. In some instances, assaying the multiple analytes occurs in the same reaction volume. Alternatively, assaying the multiple analytes occurs in separate or multiple reaction volumes.

A “target sequence” as used herein refers to a region of the genome against which one or more probes can be designed. A “target sequence” may be a coding target or a non-coding target. A “target sequence” may comprise exonic and/or non-exonic sequences. Alternatively, a “target sequence” may comprise an ultraconserved region. An ultraconserved region is generally a sequence that is at least 200 base pairs and is conserved across multiple species. An ultraconserved region may be exonic or non-exonic. Exonic sequences may comprise regions on a protein-coding gene, such as an exon, UTR, or a portion thereof. Non-exonic sequences may comprise regions on a protein-coding, non protein-coding gene, or a portion thereof.

As used herein, a probe is any polynucleotide capable of selectively hybridizing to a target sequence or its complement, or to an RNA version of either. A probe may comprise ribonucleotides, deoxyribonucleotides, peptide nucleic acids, and combinations thereof. A probe may optionally comprise one or more labels. In some embodiments, a probe may be used to amplify one or both strands of a target sequence or an RNA form thereof, acting as a sole primer in an amplification reaction or as a member of a set of primers.

“Optional” or “optionally” means that the subsequently described event or circumstance may or may not occur, and that the description includes instances where the event or circumstance occurs and instances in which it does not.

As used herein, the term “subject” refers to mammals or non-mammals. In some instances, the subject is a mammal, such as, a human, non-human primate (e.g., apes, monkeys, chimpanzees), cat, dog, rabbit, goat, horse, cow, pig, and sheep. In a preferred aspect, the subject is a human. In some instances, the subject is a patient or other individual undergoing a treatment regimen, or being evaluated for a treatment regimen (e.g., anti-AD therapy). However, in some cases, the subject is not undergoing a treatment regimen.

The methods disclosed herein often comprise assaying the expression level of a plurality of targets. The plurality of targets may include any one or more of ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093 respectively.

In some instances, the coding target and/or non-coding target is at least about 70% identical to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. Alternatively, the coding target and/or non-coding target are at least about 80% identical to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093 In some instances, the coding target and/or non-coding target are at least about 85% identical to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the coding target and/or non-coding target are at least about 90% identical to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. Alternatively, the coding target and/or non-coding target are at least about 95% identical to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

In some instances, the plurality of targets comprises at least 1 or more targets selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

In some instances, the plurality of targets comprises one or more targets comprising a sequence that comprising 10 or more consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

In some instances, the plurality of targets comprises one or more targets comprising a sequence that is complementary to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the plurality of targets comprises one or more targets comprising a sequence that is substantially complementary to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the plurality of targets comprises one or more targets comprising a sequence that is complementary to at least a portion of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

In some instances, the plurality of targets comprises one or more targets comprising a sequence that is complementary to 10 or more consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the plurality of targets comprises a sequence that is complementary to 20, 30, 40, 50, 60, 70, 80, 90, 100, 125, 150, 200, 250, 300, 350, 400, 450, 500, 600, 700, 800, 900, or 1000, 1100, 1200, 1300, or 1400 or more consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

In some instances, the plurality of targets comprises one or more targets comprising a sequence that hybridizes to a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the plurality of targets comprises one or more targets comprising a sequence that hybridizes to at least a portion of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 10 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 15 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 20 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 25 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 30, 40, 50, 60, 70, 80, 90, or 100 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to at least 150, 200, 250, 300, 350, 400, 450, 500, 600, 700, 800, 900, or 1000 consecutive nucleotides of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093. In some instances, the sequence hybridizes to a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, or a reverse complement of a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093.

The present invention provides for a probe set for diagnosing, monitoring and/or predicting a status or outcome of AD in a subject comprising a plurality of probes, wherein (i) the probes in the set detect an expression level of one or more targets selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or a combination thereof; and (ii) the expression level determines the diagnosing, monitoring and/or predicting a status or outcome of AD of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

In some instances, microarray hybridization of RNA, extracted from AD tissue samples and amplified, may yield a dataset that is then summarized and normalized by the fRMA technique. The raw expression probes are summarized and normalized into probe selection regions (“PSRs”). The cross-hybridizing PSRs, highly variable PSRs (variance above the 90th percentile), and PSRs containing more than 4 probes are removed or filtered. Following fRMA and filtration, the data can be decomposed into its principal components and an analysis of variance model is used to determine the extent to which a batch effect remains present in the first 10 principal components.

One skilled in the art understands that the nucleotide sequence of the polynucleotide probe need not be identical to its target sequence in order to specifically hybridize thereto. The polynucleotide probes of the present invention, therefore, comprise a nucleotide sequence that is at least about 65%, 70%. 75%, 80%, 85%, 90%, 95% or 100% identical to a region of the nucleic acid sequence of a target selected from the group consisting of ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 (designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093).

Methods of determining sequence identity are known in the art and can be determined, for example, by using the BLASTN program of the University of Wisconsin Computer Group (GCG) software or provided on the NCBI website. The nucleotide sequence of the polynucleotide probes of the present invention may exhibit variability by differing (e.g., by nucleotide substitution, including transition or transversion) at one, two, three, four or more nucleotides from the sequence of the coding target or non-coding target.

Other criteria known in the art may be employed in the design of the polynucleotide probes of the present invention. For example, the probes can be designed to have <50% G content and/or between about 25% and about 70% G+C content. Strategies to optimize probe hybridization to the target nucleic acid sequence can also be included in the process of probe selection.

Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

The polynucleotide probes of the present invention may range in length from about 15 nucleotides to the full length of the target. In one embodiment of the invention, the polynucleotide probes are at least about 15, 20, 25, 50, 75, 100, 125, 150, 200, 225, 250, 275, 300, 325, 350, 375 400, 450, 500, 550, 600, 650, 700, 750, 800, 850, 900, 950, 1000, 1100, 1200, 1300, or 1400 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50, 60, 70, 80, 90, or 100 nucleotides in length. Alternatively, the pairs of primers can generate an amplification product of at least 150, 200, 250, 300, 400, 500, 600, 700, 800, 900, or 1000 nucleotides in length. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a target selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or any combination thereof. In some embodiments, the primers or primer pairs, when used in an amplification reaction, specifically amplify at least a portion of a target comprising a sequence selected from the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, an RNA form thereof, a complement to either thereof, or a reverse complement to either thereof.

Exemplary polynucleotide probes or primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates.

The present invention also contemplates oligonucleotide mimetics in which both the sugar and the internucleoside linkage of the nucleotide units are replaced with novel groups. The base units are maintained for hybridization with an appropriate nucleic acid target compound. An example of such an oligonucleotide mimetic is a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

Polynucleotide probes or primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C.

One skilled in the art recognizes that it is not necessary for all positions in a given polynucleotide probe or primer to be uniformly modified. The present invention, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences which are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the invention. A variety of different coding schemes are known in the art, including fluorophores, including SCNCs, deposited metals, and RF tags.

Polynucleotides from the described target sequences may be employed as probes for detecting target sequences expression, for ligation amplification schemes, or may be used as primers for amplification schemes of all or a portion of a target sequences. When amplified, either strand produced by amplification may be provided in purified and/or isolated form.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like, and include semiconductor nanocrystals.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

Silica aerogels can also be used as substrates, and can be prepared by methods known in the art. Aerogel substrates may be used as free standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. The surface can be optically transparent and can have surface Si—OH functionalities, such as those found on silica surfaces.

The substrate and/or its optional surface can be chosen to provide appropriate characteristics for the synthetic and/or detection methods used. The substrate and/or surface can be transparent to allow the exposure of the substrate by light applied from multiple directions. The substrate and/or surface may be provided with reflective “mirror” structures to increase the recovery of light.

The substrate and/or its surface is generally resistant to, or is treated to resist, the conditions to which it is to be exposed in use, and can be optionally treated to remove any resistant material after exposure to such conditions.

The substrate or a region thereof may be encoded so that the identity of the sensor located in the substrate or region being queried may be determined. Any suitable coding scheme can be used, for example optical codes, RFID tags, magnetic codes, physical codes, fluorescent codes, and combinations of codes.

Preparation of Probes and Primers

The polynucleotide probes or primers of the present invention can be prepared by conventional techniques well-known to those skilled in the art. For example, the polynucleotide probes can be prepared using solid-phase synthesis using commercially available equipment. As is well-known in the art, modified oligonucleotides can also be readily prepared by similar methods. The polynucleotide probes can also be synthesized directly on a solid support according to methods standard in the art. This method of synthesizing polynucleotides is particularly useful when the polynucleotide probes are part of a nucleic acid array.

Polynucleotide probes or primers can be fabricated on or attached to the substrate by any suitable method, for example the methods described in U.S. Pat. No. 5,143,854, PCT Publ. No. WO 92/10092, U.S. patent application Ser. No. 07/624,120, filed Dec. 6, 1990 (now abandoned), Fodor et al., Science, 251: 767-777 (1991), and PCT Publ. No. WO 90/15070). Techniques for the synthesis of these arrays using mechanical synthesis strategies are described in, e.g., PCT Publication No. WO 93/09668 and U.S. Pat. No. 5,384,261. Still further techniques include bead based techniques such as those described in PCT Appl. No. PCT/US93/04145 and pin based methods such as those described in U.S. Pat. No. 5,288,514. Additional flow channel or spotting methods applicable to attachment of sensor polynucleotides to a substrate are described in U.S. patent application Ser. No. 07/980,523, filed Nov. 20, 1992, and U.S. Pat. No. 5,384,261.

Alternatively, the polynucleotide probes of the present invention can be prepared by enzymatic digestion of the naturally occurring target gene, or mRNA or cDNA derived therefrom, by methods known in the art.

Samples

Diagnostic samples for use with the systems and in the methods of the present invention comprise nucleic acids suitable for providing RNAs expression information. In principle, the biological sample from which the expressed RNA is obtained and analyzed for target sequence expression can be any material suspected of comprising AD tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising AD tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, blood, plasma, serum, cerebrospinal fluid and organs.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Hely solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

The samples can be obtained at any time. In some instances, multiple samples are obtained from the subject. In some instances, the samples are obtained prior to diagnosis, after diagnosis, prior to prognosis, after prognosis, prior to administration of a therapeutic regimen, during administration of a therapeutic regimen, after administration of a therapeutic regimen, prior to modification of a therapeutic regimen, during modification of a therapeutic regimen, after modification of a therapeutic regimen, or a combination thereof. In some instances, the samples are obtained 1, 2, 3, 4, 5, 6, 7, 8, 9, 10, 11, 12, 13, or 14 days; 1, 2, 3, 4, 5, 6, 7, 8, 9, 10, 11, 12, 13, or 14 weeks; 1, 2, 3, 4, 5, 6, 7, 8, 9, 10, 11, 12, 13, or 14 months; or 1, 2, 3, 4, 5, 6, 7, 8, 9, 10, 11, 12, 13, or 14 years prior to diagnosis, after diagnosis, prior to prognosis, after prognosis, prior to administration of a therapeutic regimen, during administration of a therapeutic regimen, after administration of a therapeutic regimen, prior to modification of a therapeutic regimen, during modification of a therapeutic regimen, after modification of a therapeutic regimen, or a combination thereof. In some instances, the samples are obtained about every 1, 2, 3, 4, 5, 6, 7, 8, 9, 10, 11, 12, 13, or 14 days; 1, 2, 3, 4, 5, 6, 7, 8, 9, 10, 11, 12, 13, or 14 weeks; 1, 2, 3, 4, 5, 6, 7, 8, 9, 10, 11, 12, 13, or 14 months; or 1, 2, 3, 4, 5, 6, 7, 8, 9, 10, 11, 12, 13, or 14 years prior to diagnosis, after diagnosis, prior to prognosis, after prognosis, prior to administration of a therapeutic regimen, during administration of a therapeutic regimen, after administration of a therapeutic regimen, prior to modification of a therapeutic regimen, during modification of a therapeutic regimen, after modification of a therapeutic regimen, or a combination thereof. In some instances, two or more samples are obtained from the subject. In some instances, three or more samples are obtained from the subject. In some instances, four or more samples are obtained from the subject. In some instances, five or more samples are obtained from the subject. In some instances, at least 6, 7, 8, 9, 10, 11, 12, 13, 14, 15, 20, 25, 30, 35, 40, 45, 50, 55, 60, 70, 80, 90, or 100 samples are obtained from the subject.

DNA and RNA Extraction

DNA can be extracted and purified from biological samples using any suitable technique. A number of techniques for DNA extraction and/or purification are known in the art, and several are commercially available (e.g., ChargeSwitch®, MELT™ total nucleic acid isolation system, MagMAX™ FFPE total nucleic acid isolation kit, MagMAX™ total nucleic acid isolation kit, QIAamp DNA kit, Omni-Pure™ genomic DNA purification system, WaterMaster™ DNA purification kit). Reagents such as DNAzol® and TRI Reagent® can also be used to extract and/or purify DNA. DNA can be further purified using Proteinase K and/or RNAse.

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

DNA and/or RNA concentrations can be made using a Nanodrop ND-1000 spectrophotometer (Nanodrop Technologies, Rockland, Del.). DNA and/or RNA can be extracted from frozen tissues, whole blood, hair, sperm, bones, nails, tissues, blood stains, saliva, buccal (cheek) swabs, epithelial cells, and urine.

Kits

Kits for performing the desired method(s) are also provided. In some instances, the kit comprises a container or housing for holding the components of the kit, one or more vessels containing one or more nucleic acid(s), and optionally one or more vessels containing one or more reagents. The reagents include those described herein, and those reagents useful for performing the methods described herein, including amplification reagents, purification reagents, binding reagents, and may include one or more probe sets, probes, primers or primer pairs, enzymes (including polymerases and ligases), intercalating dyes, labeled probes, labels that can be incorporated into amplification products, and labels that can be attached or hybridized to a target or probe disclosed herein. In some embodiments, the kit comprises primers or primer pairs specific for those subsets and combinations of target sequences described herein. In some instances, the kit comprises at least two, three, four or five primers or pairs of primers suitable for isolating, amplifying, hybridizing, sequencing, and/or quantifying one or more targets disclosed herein. In some embodiments, the kit comprises from five to fifty primers or pairs of primers suitable for isolating, amplifying, hybridizing, sequencing, and/or quantifying one or more targets disclosed herein. In some embodiments, the kit comprises at least 10, 15, 20, 25, 30, 35, 40, 45, 50, 55, 60, 65, 70, 75, 80, 85, 90, 95, 100, 110, 120, 130, 140, or 150 primers or pairs of primers suitable for isolating, amplifying, hybridizing, sequencing, and/or quantifying one or more targets disclosed herein.

In some embodiments, the primers or primer pairs of the kit, when used in an amplification reaction, specifically amplify a non-coding target, coding target, or non-exonic target described herein, at least a portion of a nucleic acid sequence designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a nucleic acid sequence corresponding to a target selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, an RNA form thereof, or a complement to either thereof.

The kit may include a plurality of such primers or primer pairs which can specifically amplify a corresponding plurality of different amplify nucleic acids depicted in one of the sequences designated by GenBank Accession numbers NM_140462, NM_638, NM_4693, NM_10842; NM_55655, NM_5354, NM_51151, NM_6909, NM_10382, NM_91975, NM_79924, NM_401491, NM_4502, NM_5743, NM_1244, NM_9427, NM_80864, NM_56884, and NM_64093, a nucleic acid sequence corresponding to a target selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or any combination thereof, RNA forms thereof, or complements thereto. At least two, three, four or five primers or pairs of primers suitable for selectively amplifying the same number of target sequence-specific polynucleotides can be provided in kit form. In some embodiments, the kit comprises from five to fifty primers or pairs of primers suitable for amplifying the same number of target sequence-representative polynucleotides of interest. In some instances, the targets are selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or any combination thereof.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic AD.

The probe sets, probes, primers or primer pairs may be provided in an array format, and thus an array or microarray may be included in the kit. The kit optionally may be certified by a government agency for use in prognosing the disease outcome of AD patients and/or for designating a treatment modality.

In some instances, the probe sets, probes, primers or primer pairs are attached to a solid support. A solid support comprises any solid platform to which a probe set, probe, primer, or primer pair can be attached. Examples of solid supports include, but are not limited to, beads, plates, multiwall plates, CPG particles, or MPPS materials. In some instances, the bead is a streptavidin bead, agarose bead, magnetic bead, Dynabeads®, MACS® microbead, antibody conjugated bead (e.g., anti-immunoglobulin microbead), protein A conjugated bead, protein G conjugated bead, protein A/G conjugated bead, protein L conjugated bead, oligo-dT conjugated bead, silica bead, silica-like bead, anti-biotin microbead, anti-fluorochrome microbead, or BcMag™ Carboxy-Terminated Magnetic Bead. In some instances, the plate is a MSD multi-array plate, MSD Multi-Spot® plate, microplate, ProteOn microplate, AlphaPlate, DELFIA plate, IsoPlate, or LumaPlate. In some instances, the CPG material has a pore size of 500, 1000, 1500, 2000, or 3000 Å. In some instances, the solid support is porous. In some instances, the solid support is macroporous.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

Devices

Devices useful for performing methods of the invention are also provided. The devices can comprise means for characterizing the expression level of a target sequence of the invention, for example components for performing one or more methods of nucleic acid extraction, amplification, and/or detection. Such components may include one or more of an amplification chamber (for example a thermal cycler), a plate reader, a spectrophotometer, luminometer, fluorometer, electrophoresis apparatus, capillary electrophoresis apparatus, chip reader, plate reader, microarray reader, computer, and or robotic sample handling components. These components ultimately can obtain data that reflects the expression level of the target sequences used in the assay being employed.

The devices may include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Exemplary excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices typically comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory that contains a sample designation and a record of expression levels obtained from the sample. Linkage of the results to the sample can also include a linkage to a particular sample receptacle in the device, which is also linked to the sample identity.

The devices also comprise a means for correlating the expression levels of the target sequences being studied with a prognosis of disease outcome. Such means may comprise one or more of a variety of correlative techniques, including lookup tables, algorithms, multivariate models, and linear or nonlinear combinations of expression models or algorithms. The expression levels may be converted to one or more likelihood scores, reflecting a likelihood that the patient providing the sample may exhibit a particular disease outcome. The models and/or algorithms can be provided in machine readable format and can optionally further designate a treatment modality for a patient or class of patients.

The device also comprises output means for outputting the disease status, prognosis and/or a treatment modality. Such output means can take any form which transmits the results to a patient and/or a healthcare provider, and may include a monitor, a printed format, or both. The device may use a computer system for performing one or more of the steps provided.

The methods disclosed herein may also comprise the transmission of data/information. For example, data/information derived from the detection and/or quantification of the target may be transmitted to another device and/or instrument. In some instances, the information obtained from an algorithm may also be transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc).

Transmission of the data/information may comprise digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. The data may be represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

Amplification and Hybridization

Following sample collection and nucleic acid extraction, the nucleic acid portion of the sample that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid (e.g., RNA, DNA). In some instances, the nucleic acid is an expressed RNA. An amplification product can be RNA or DNA, and may include a complementary strand to the target sequence. In some instances, DNA amplification products are produced initially through reverse transcription and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), rolling circle amplification, ribozyme-based methods, self sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation'm FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAC1 can magnetic beads, Agencourt Biosciences).

Multiple biomarkers (e.g., targets) can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the invention. The target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

Reverse Transcription for QRT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

TaqMan® Gene Expression Analysis

TaqMan®RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

Classification Arrays

In some instances, a probe set or probes derived herein is provided in an array format. In some instances, an “array” is a spatially or logically organized collection of polynucleotide probes. An array comprising probes specific for a coding target, non-coding target, or a combination thereof may be used. Alternatively, an array comprising probes specific for two or more of the targets selected from ASB9, BIK, C7orfl6, NDP, NLRP2, PLP1, SLC45A2, TBX2, TUBB4, ZNF300, ADM2, FLJ35024, MT2A, PTGS2, ABCC2, ECEL1, EGFL8, FSTL5, and SMOC1 or any combination thereof. In some instances, expression of these target sequences or targets are detected alone or in combination with other transcripts or targets. Examples of other transcripts or targets include, but are not limited to, any of the transcripts and/or targets disclosed in U.S. patent application Ser. Nos. 13/254,571 and 13/258,429. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment, the array is a chip.

Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit AD. The likelihood score can be used to distinguish these disease states. The models and/or algorithms can be provided in machine readable format, and may be used to correlate expression levels or an expression profile with a disease state, and/or to designate a treatment modality for a patient or class of patients.

In some instances, assaying the expression level for a plurality of targets comprises the use of an algorithm. Array data can be managed, classified, and analyzed using techniques known in the art. Assaying the expression level for a plurality of targets can comprise probe set modeling and/or data pre-processing. Probe set modeling and/or data pre-processing can be derived using the Robust Multi-Array (RMA) algorithm or variants GC-RMA, fRMA, Probe Logarithmic Intensity Error (PLIER) algorithm or variant IterPLIER. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

Alzheimer's Disease

Although Alzheimer's disease develops differently for every individual, there are many common symptoms. Early symptoms are often mistakenly thought to be ‘age-related’ concerns, or manifestations of stress. In the early stages, the most common symptom is difficulty in remembering recent events. When AD is suspected, the diagnosis is usually confirmed with tests that evaluate behavior and thinking abilities, often followed by a brain scan if available, however, examination of brain tissue is required for a definitive diagnosis. As the disease advances, symptoms can include confusion, irritability, aggression, mood swings, trouble with language, and long-term memory loss. As the sufferer declines they often withdraw from family and society. Gradually, bodily functions are lost, ultimately leading to death. Since the disease is different for each individual, predicting how it will affect the person is difficult. AD develops for an unknown and variable amount of time before becoming fully apparent, and it can progress undiagnosed for years. On average, the life expectancy following diagnosis is approximately seven years. Fewer than three percent of individuals live more than fourteen years after diagnosis.

Therapeutic Regimens

Five medications are currently used to treat the cognitive problems of AD: four are acetylcholinesterase inhibitors (tacrine, rivastigmine, galantamine and donepezil) and the other (memantine) is an NMDA receptor antagonist. The benefit from their use is small. No medication has been clearly shown to delay or halt the progression of the disease.

Reduction in the activity of the cholinergic neurons is a well-known feature of Alzheimer's disease. Acetylcholinesterase inhibitors are employed to reduce the rate at which acetylcholine (ACh) is broken down, thereby increasing the concentration of ACh in the brain and combating the loss of ACh caused by the death of cholinergic neurons. There is evidence for the efficacy of these medications in mild to moderate Alzheimer's disease, and some evidence for their use in the advanced stage. Only donepezil is approved for treatment of advanced AD dementia. The use of these drugs in mild cognitive impairment has not shown any effect in a delay of the onset of AD. The most common side effects are nausea and vomiting, both of which are linked to cholinergic excess. These side effects arise in approximately 10-20% of users and are mild to moderate in severity. Less common secondary effects include muscle cramps, decreased heart rate (bradycardia), decreased appetite and weight, and increased gastric acid production.

Glutamate is a useful excitatory neurotransmitter of the nervous system, although excessive amounts in the brain can lead to cell death through a process called excitotoxicity which consists of the overstimulation of glutamate receptors. Excitotoxicity occurs not only in Alzheimer's disease, but also in other neurological diseases such as Parkinson's disease and multiple sclerosis. Memantine (brand names Akatinol) is a noncompetitive NMDA receptor antagonist first used as an anti-influenza agent. It acts on the glutamatergic system by blocking NMDA receptors and inhibiting their overstimulation by glutamate. Memantine has been shown to be moderately efficacious in the treatment of moderate to severe Alzheimer's disease. Its effects in the initial stages of AD are unknown. Reported adverse events with memantine are infrequent and mild, including hallucinations, confusion, dizziness, headache and fatigue. The combination of memantine and donepezil has been shown to be “of statistically significant but clinically marginal effectiveness”.

As used herein, the term “test agent” means any compound or agent that is being examined for the ability to reduce treat Alzheimer's disease. A test agencan be any type of molecule, including, for example a peptide, a polynucleotide (including antisense or RNAi), an antibody, a glycoprotein, a carbohydrate, a small organic molecule, or a peptidomimetic.

As used herein, a “control” or “normal” is any sample taken from a subject of similar species that is considered healthy or otherwise not suffering from AD or a related disorder.

The test agent as disclosed herein can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. A pharmaceutical composition also can be administered to the site of a pathologic condition, for example, intravenously orintra-arterially into a blood vessel supplying a tissue or organ comprising retrovirus infected cells

Human NPCs are an important cell type to study in context of AD, due to deficits in adult neurogenesis and newborn neuron survival seen in AD mouse models and potentially human patients. There might also be developmental components of FAD that could be reflected in NPCs. In addition, NPCs are at least somewhat more homogenous than the wide variety of neurons produced by current general neuronal differentiation protocols, which might allow better cross comparisons between control and FAD cells. To date, no study has addressed human iPSC-derived NPCs in the context of Alzheimer's disease and thus, here is disclosed a PSEN1 iPSC model that was used to interrogate potential alterations in FAD NPCs.

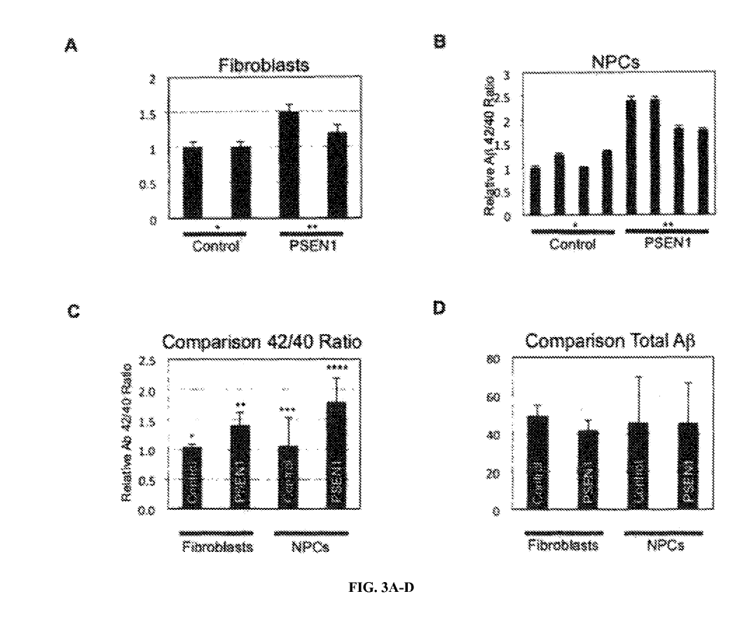

Potential differences in Aβ processing were addresses first, the most critical component of known FAD pathology. Similar to a recent study of transdifferentiated PSEN1 neurons, PSEN1 NPCs have been shown to have higher Aβ42/Aβ40 ratios than equivalent control cells and in a greater magnitude than as fibroblasts (FIG. 3). This is the first study of human NPCs that addresses pathogenic proteolytic processing of endogenous APP. Human NPCs therefore produce their own elevated supply of Aβ42:40 that may affect their developmental potential and survival.

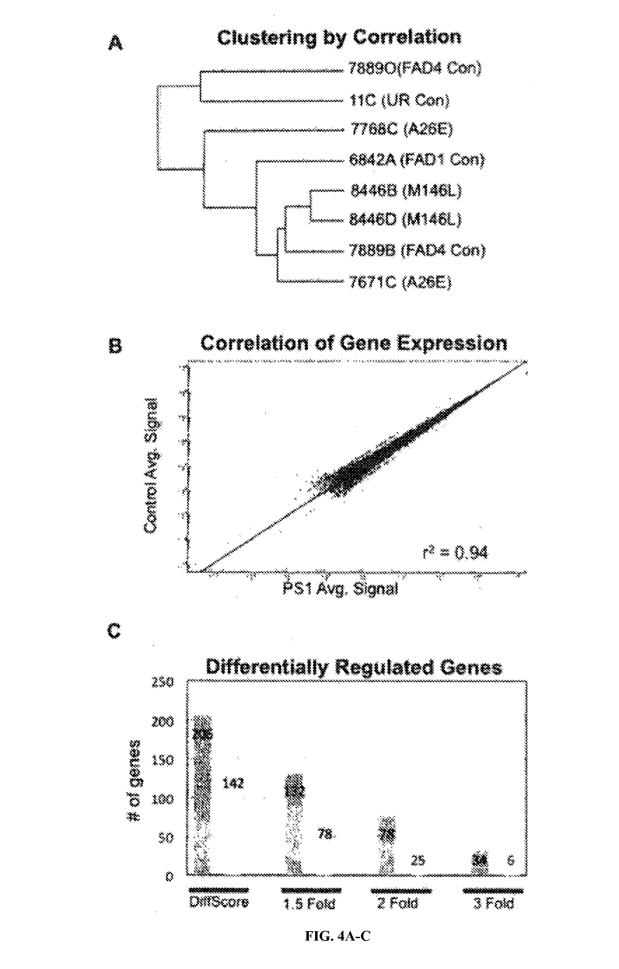

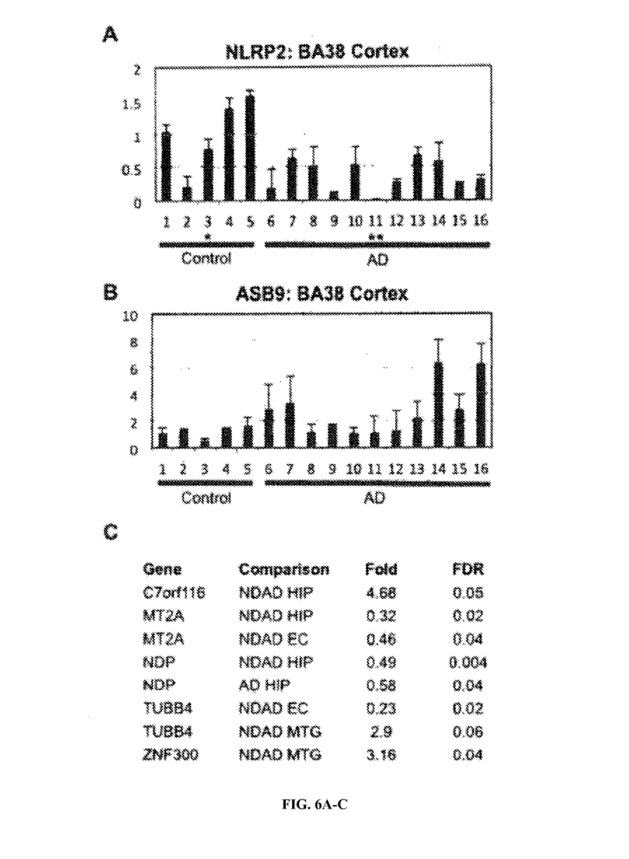

Having established the validity of the FAD iPSC model, it was then investigated whether novel molecules potentially important for AD could be identified. Towards this end, molecular profiling experiments were performed utilizing both undifferentiated and 14-day differentiated iPSCs from the core set (FIG. 4). This led to the identification of 14 genes with altered expression in PSEN1 NPCs (Table 2). Several of these also showed significant differences in late onset brains by either qPCR or data mining methods (FIG. 6).

Three targets were looked at in further detail: NLRP2, ASB9, and NDP. NLRP2 was also the only gene differentially regulated in undifferentiated iPSCs. Control line 6842A was an outlier regarding NLRP2 expression. Interestingly, the gene is located at the breakpoint of the balanced location in this line. It is likely that this chromosomal alteration is responsible for NLRP2 expression level differences, although future studies will need to confirm this is the case. If true, such misregulation would be expected to have consequences for this balanced translocation. It is intriguing that NLRP2 expression was decreased in late onset AD brains. One possibility is that the cells that express NLRP2 at high levels (and thus would be predicted to have an increased pro-inflammatory response) might be more vulnerable in AD. On the other hand, ASB9, and E3-ligase (which inhibits mitochondrial function) was shown to be upregulated in brains from some late onset AD patients.

NDP is a particularly interesting target in the context of the proposal that FAD might have developmental consequences, in that 30% of patients with Norrie Disease, a disorder caused by mutation of NDP, have CNS deficits. In addition, promotion of Wnt signaling and inhibition of TGFβ signaling by norrin should enhance adult neurogenesis by driving proliferation/NeuroD1 expression, and inhibiting astrocyte fate respectively. Thus the reduction of NDP in the hippocampus of late onset AD brains would be predicted to decrease neurogenic potential. Future studies incorporating gain and loss of function of NDP should help clarify NDP's potential role in neurogenesis and Alzheimer disease.

### EXEMPLARY EMBODIMENTS

Disclosed herein is a method of diagnosing, prognosing, determining progression of Alzheimer's disease, predicting a therapeutic regimen or predicting benefit from therapy in a subject having Alzheimer's disease comprising: assaying an expression level of a plurality of targets in a sample from the subject, wherein the plurality of targets comprises one or more targets maybe NLRP2, ASB9, BIK, C7orfl6, ZNF300 or ADM2 or any combination thereof; and diagnosing, prognosing, determining progression of AD, predicting a therapeutic regimen or predicting benefit from therapy in a subject having AD based on the expression levels of the plurality of targets. In one embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In an additional embodiment, ASB9, BIK, C7orfl6, NDP, NLRP2, PLP1, SLC45A2, TBX2, TUBB4 and/or ZNF300 are upregulated. In another embodiment, ADM2, FLJ35024, MT2A and/or PTGS2 are downregulated. In some embodiments, the plurality of targets comprises two or more targets. In some embodiments, the method further comprises amplifying the plurality of targets, conducting a multiplexed reaction, sequencing the plurality of targets, quantifying the plurality of targets, hybridizing a label to a plurality of targets, amplifying the plurality of targets, reverse transcribing the plurality of targets, or hybridizing a probe to the plurality of targets, In some embodiments, the probe or plurality of targets is labeled. In some embodiments, the method further comprises attaching the plurality of targets to a solid support. In some embodiments, the method further comprises attaching a probe or probe set to a solid support. In some embodiments, the solid support is an array, a bead, a plate, or microwell plate. In some instances, the expression level determines the AD status of the subject with at least 40%, 45%, 50%, 60%, 65%, 70%, 75%, 80%, 85%, 90%, 95% or 100% specificity.

Also disclosed herein is a system for analyzing Alzheimer's disease in a subject, comprising: a probe set comprising a plurality of target sequences, wherein the plurality of target sequences hybridizes to more than one target which may be NLRP2, ASB9, BIK, C7orfl6, ZNF300 and ADM2 or any combination thereof; a plurality of probes that hybridize to the plurality of target sequences; a sample from a subject having Alzheimer's disease; a computer model or algorithm for analyzing an expression level and/or expression profile of the targets hybridized to the probes in a sample from a subject having Alzheimer's disease. In some embodiments, the system further comprises an electronic memory for capturing and storing an expression profile; a computer-processing device, optionally connected to a computer network; a software module executed by the computer-processing device to analyze an expression profile; a software module executed by the computer-processing device to compare the expression profile to a standard or control; a software module executed by the computer-processing device to determine the expression level of the target; a machine to isolate the target or the probe from the sample; a machine to sequence the target or the probe; a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises two or more targets. In additional embodiments, the target further comprises NDP, PLP1, SLC45A2, TBX2, TUBB4, FLJ35024, MT2A, PTGS2, ABCC2, ECEL1, EGFL8, FSTL5, or SMOC1 or any combination thereof.

Further disclosed herein is a method of treating a subject with Alzheimer's disease comprising: obtaining an expression profile of a plurality of targets from a sample from a subject; determining the subject has Alzheimer's disease by comparing the expression profile from the patient to an expression profile from a control or standard; and administering treatment to the subject, wherein the plurality of targets comprises one or more of the following targets NLRP2, ASB9, BIK, C7orfl6, ZNF300 and ADM2 or any combination thereof. In an embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In some embodiments, ASB9, BIK, C7orfl6, NDP, NLRP2, PLP1, SLC45A2, TBX2, TUBB4 and/or ZNF300 are upregulated. In additional embodiments, ADM2, FLJ35024, MT2A and/or PTGS2 are downregulated. In further embodiments, the treatment is an acetylcholinesterase inhibitor or a NMDA receptor antagonist. In another embodiment, the treatment maybe tacrine, rivastigmine, galantamine, donepezil or memantine.

Also disclosed herein is a method of identifying an agent for the treatment of Alzheimer's disease comprising: contacting a cell with a test agent, wherein the expression of a plurality of targets in the cell is consistent with Alzheimer's disease; determining the expression of the plurality of targets from the cell; comparing the expression of the plurality of targets from the cell to a control or standard; and determining that the expression of the plurality of targets is not consistent with Alzheimer's disease, wherein the plurality of targets comprises one or more of the following targets NLRP2, ASB9, BIK, C7orfl6, ZNF300 and ADM2 or any combination thereof. In an embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In one embodiment, up regulation of ASB9, BIK, C7orfl6, NDP, NLRP2, PLP1, SLC45A2, TBX2, TUBB4 and/or ZNF300 is consistent with Alzheimer's disease. In a further embodiment, downregulation of ADM2, FLJ35024, MT2A and/or PTGS2 is consistent with Alzheimer's disease.

Further disclosed herein is a kit for analyzing Alzheimer's disease, comprising: a probe set comprising a plurality of target sequences, wherein the plurality of target sequences hybridizes to more than one target of the following NLRP2, ASB9, BIK, C7orfl6, ZNF300 and ADM2 or any combination thereof; and a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. altering a treatment regimen. In a further embodiment, the target further comprises NDP, PLP1, SLC45A2, TBX2, TUBB4, FLJ35024, MT2A, PTGS2, ABCC2, ECEL1, EGFL8, FSTL5, or SMOC1 or any combination thereof.

Also disclosed herein is a system for analyzing Alzheimer's disease comprising: a computer processing device for determining an expression profile for a plurality of targets; an electronic memory device for capturing and storing an expression profile; a software module; a machine to isolate target or the probe from the sample; a machine to sequence the target or the probe; a machine to amplify the target or the probe; a computer model or algorithm for analyzing an expression level and/or expression profile of the target hybridized to the probe in a sample from a subject suffering from AD; and an output providing the analysis. In one embodiment, the a plurality of targets comprises one or more of the following targets NLRP2, ASB9, BIK, C7orfl6, ZNF300 and ADM2 or any combination thereof. In another embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In an embodiment, the software module executed by the computer-processing device analyzes an expression profile. In some embodiments, the software compare executed by the computer-processing devices the expression profile to a standard or control; determines the expression level of the target; transmits an analysis of the expression profile to the subject or a medical professional treating the subject. In another embodiment, the system further comprises a label that specifically binds to the target, the probe, or a combination thereof.

Additionally disclosed herein is a method for analyzing Alzheimer's disease comprising: a computer processing device for determining an expression profile for a plurality of targets; an electronic memory device for capturing and storing an expression profile; a software module; a machine to isolate target or the probe from the sample; a machine to sequence the target or the probe; a machine to amplify the target or the probe; a computer model or algorithm for analyzing an expression level and/or expression profile of the target hybridized to the probe in a sample from a subject suffering from AD; and an output providing the analysis. In one embodiment, the a plurality of targets comprises one or more of the following targets NLRP2, ASB9, BIK, C7orfl6, ZNF300 and ADM2 or any combination thereof. In another embodiment, the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof. In an embodiment, the software module executed by the computer-processing device analyzes an expression profile. In some embodiments, the software compare executed by the computer-processing devices the expression profile to a standard or control; determines the expression level of the target; transmits an analysis of the expression profile to the subject or a medical professional treating the subject. In another embodiment, the method further comprises a label that specifically binds to the target, the probe, or a combination thereof.

Disclosed herein is a method of diagnosing, prognosing, determining progression of Alzheimer's disease, predicting a therapeutic regimen or predicting benefit from therapy in a subject having Alzheimer's disease comprising assaying an expression level a protein encoded by at least one or more of a plurality of targets in a sample from the subject, wherein the plurality of targets comprises one or more targets selected from NLRP2, ASB9, BIK, C7orfl6, ZNF300 or ADM2 or any combination thereof; and diagnosing, prognosing, determining progression AD, predicting a therapeutic regimen or predicting benefit from therapy in a subject having AD based on the protein expression levels of the plurality of targets. In one embodiment, the plurality of targets further comprises targets selected from one or more of NDP, PLP1, SLC45A2, TBX2, TUBB4, FLJ35024, MT2A, PTGS2, ABCC2, ECEL1, EGFL8, FSTL5, or SMOC1 or any combination thereof. In an additional embodiment, ASB9, BIK, C7orfl6, NDP, NLRP2, PLP1, SLC45A2, TBX2, TUBB4 and/or ZNF300 are upregulated. In another embodiment, ADM2, FLJ35024, MT2A and/or PTGS2 are downregulated. In some embodiments, the plurality of targets comprises two or more targets. The protein expression level may be determined by routine laboratory methods. These methods include, but are not limited to, ELISA, RIA and FACs analysis.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Generation of iPSC Lines

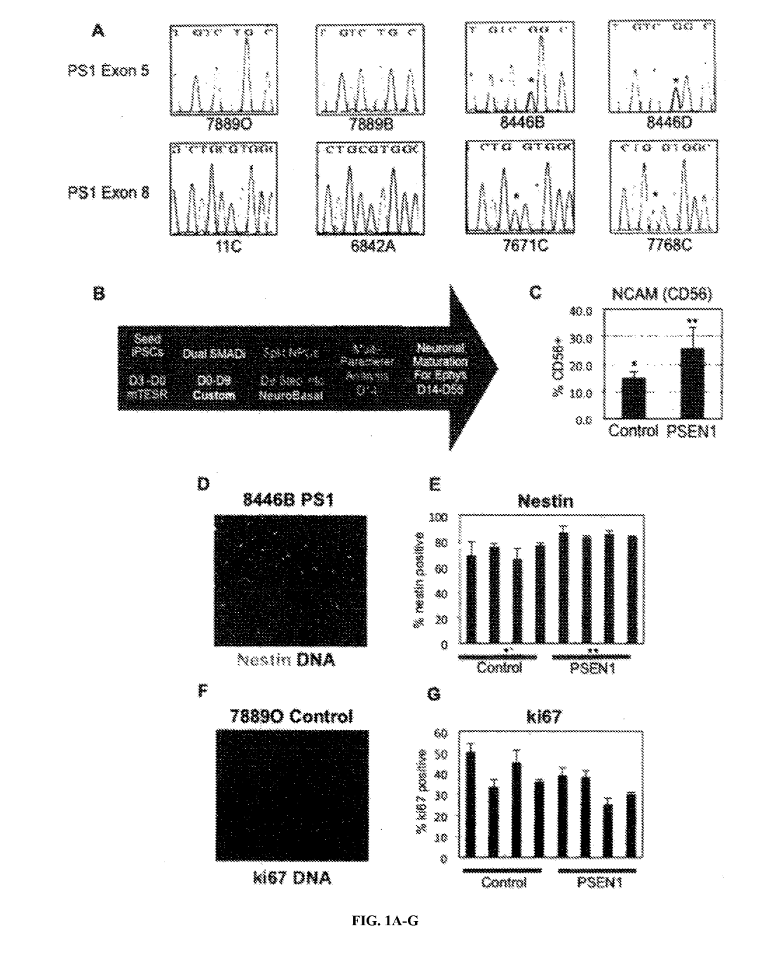

In order to create PSEN1 mutant and wild-type control iPSC lines, established fibroblast lines were obtained from the cell bank repository at the Coriell Institute (Camden, N.J.). Non-EBV transformed fibroblast lines were selected from the “Canadian” (FAD1, A246E PS1 mutation) and the “Italian” (FAD4, M146L PS1 mutation) EOFAD kindreds. Heterozygosity in the PSEN1 locus was confirmed in AD patients for fibroblasts and subsequently derived iPSCs via sequencing (FIG. 1A). Fibroblast lines were reprogrammed using four high-titer retroviral constructs prepared by the Harvard Gene Therapy Core Facility that encoded human Oct4, KLF4, SOX2 and c-Myc, respectively. iPSC colonies were initially selected by morphology, passaged several times to remove transformed cells, and expanded before characterization.

### Example 2

### Characterization of iPSCs

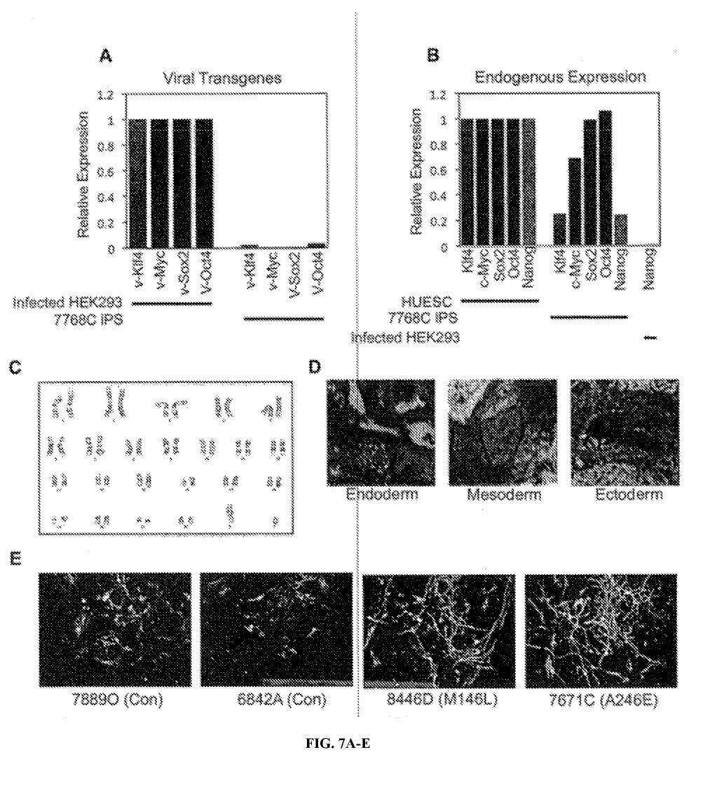

After iPSCs were expanded to multi-well format, they were characterized using a variety of quality control assays. Initial characterization included the presence of alkaline-phosphatase (AP) enzymatic activity, immunostaining for pluripotency markers, and qPCR for both endogenous pluripotent markers and viral transgene silencing. An example of initial characterization of one line (7768C) is shown in FIG. 7. Cell lines with insufficient transgene silencing were not further analyzed.

### Example 3

### Selection and Further Characterization of Core Set of iPSC Lines

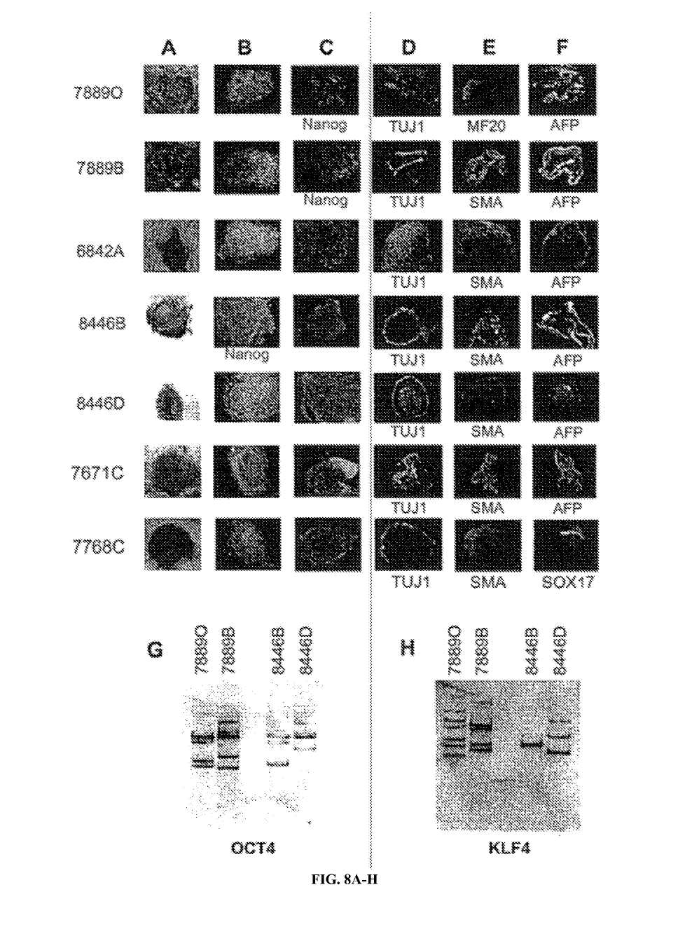

Eight iPSC lines were selected, including one unrelated control iPSC line 11C to serve as a core set for the majority of the experiments (Table 1, FIG. 7, 8). All data utilizing the core set shows the same order of cell lines as in Table 1. The best transgene shutoff and endogenous expression of stem cell genes were used as the main criteria in clone selection. In addition, core set candidates were also karyotyped (e.g. FIG. 7C) and fingerprinted (Cell Line Genetics; data not shown) to ensure that they matched the parental fibroblast line. Unfortunately, five iPSC clones from two patients from the FAD1 family harbored chromosomal mutations of various types. Since a karyotypically normal iPSC line could not be obtained from the FAD1 family, it was decided to use clone 6842A that had a balanced translocation, t(17(q22.3), 19(q13.4)). Many individuals harbor balanced translocations without issue. In addition, for one FAD4 control individual (7889) and one FAD4 AD patient (8446), two clones for further study were selected, in order to test for the possible effects of random transgene insertion. Determination that iPSC clones from each patient were independent as defined by different viral integration sites was determined by Southern blotting (FIG. 8). Integration events were analyzed for two different transgenes: Oct4 and Klf4.

In vitro pluripotency of core lines was demonstrated by undirected differentiation of iPSCs into embryoid bodies and subsequent immunostaining of frozen sections for germ-layer specific markers for each of the three developmental germ layers (FIG. 8). For cell line 7768C, it was also established in vivo pluripotency via subcutaneous injection of undifferentiated iPSCs within a matrigel matrix into the dorsal flank of NSG immune-compromised mice (Jackson Laboratory). As shown in FIG. 7, the ability to form three germ layers was assessed using hematoxylin and eosin (H&E) staining of paraffin-embedded sections of resulting teratomas. Observed tissues include: glandular epithelia, indicating the presence of endoderm; bone and cartilage, indicating differentiation of mesoderm lineages; and neural epithelia, including areas with retinal pigmented epithelium, indicating competence of differentiation towards ectodermal lineages.

TABLE 1
Core set of iPSC Lines

| Line | Family | Sex | Age | PSEN1 | APOE |
|---|---|---|---|---|---|
| 7889O | FAD4 | M | 18 | WT | ε3/ε3 |
| 7889B | FAD4 | M | 18 | WT | ε3/ε3 |
| 11C | N/A | M | 36 | WT | ε3/ε3 |
| 6842A | FAD1 | M | 75 | WT | ε3/ε3 |
| 8446B | FAD4 | M | 38 | M146L | ε3/ε3 |
| 8446D | FAD4 | M | 38 | M146L | ε3/ε3 |
| 7671C | FAD1 | M | 44 | A26E | ε3/ε3 |
| 7768C | FAD1 | F | 31 | A26E | ε3/ε4 |

### Example 4

### Analysis of APOE Genotype

APOE isotype is the most common identified risk factor associated with late-onset AD and could potentially influence observed phenotypes. APOE genotype was determined by standard restriction fragment length polymorphism method. Seven of 8 lines in the core set are APOE ε3/ε3, the reference genotype (Table 1). One line, 7768C is APOE ε3/ε4, and thus harbors both one PS1 A246E early-onset familial AD deterministic allele and one late-onset AD risk allele.

### Example 5

### Neuronal Differentiation of iPSC Lines

In order to investigate PSEN1 and control NPCs, cells were differentiated by a monolayer method and analyzed cells at different time points during this process. As shown in FIG. 1B in cartoon form, iPSCs were plated as single cells, allowed to recover, and subsequently neuralized by inhibition of both branches of TGFβ signaling pathways (dual-SMAD inhibition,). Inhibition of TGFβ pathways is sufficient to induce anterior neural fates from pluripotent cells. To look at a mixture of mostly NPCs and a minority of early-born neurons, the cell lines were assessed at Day 14 post dual-SMAD inhibition. There was a significant difference in the amount of CD56+ (NCAM) PSEN1 cells as compared to control cells as measured by flow cytometry (FCM) of live cells as determined by forward scatter plot (26% vs. 15%, p=0.02, Student's t-Test, FIG. 1C). Although CD56+ expression is often used to identify neuronal populations by FCM, it is also expressed in some nestin-positive progenitors. At day 14 of neuronal differentiation there were no NeuN+ cells, a more mature neuronal marker. However, there were small patches of Tuj1+ cells with more complex neurite morphology, which appeared to be more prevalent on average in PSEN1 cells (FIG. 7).

The largest proportion of cells at this time point was nestin-positive NPCs (FIG. 1D,E). PSEN1 lines had a small but statistically significant increase in the percentage of nestin-positive cells compared to control lines (79% vs. 71%, p=0.012, Student's t-Test). A significant proportion of cells were in cell cycle as measured by Ki67 staining (FIG. 1F,G; average for 8 lines: 33%), which was not statistically different between PSEN1 genotypes.

### Example 6

### NPCs have the Capacity to Make Electrically-Active Neurons

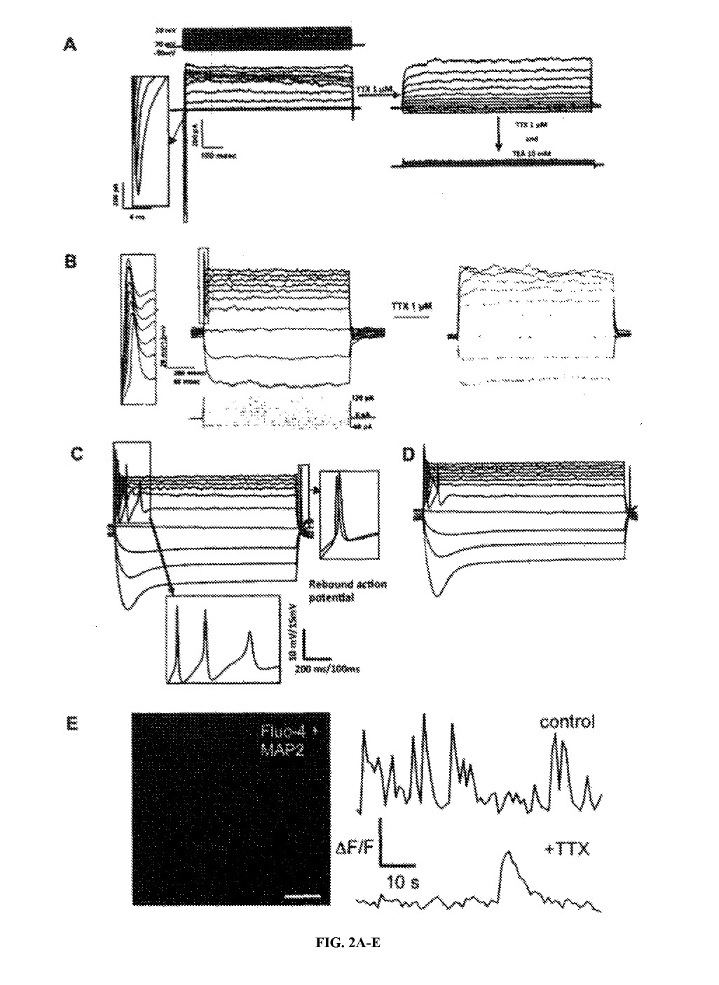

To establish that the NPCs were capable of making mature neurons that had electrical signaling properties similar to primary neurons. Thus, electrophysiogical properties were recorded from 375 cells that had been neuronally differentiated 35 to 55 days from control iPSC line 7889O and 87 cells from PS1 iPSC line 8446B. Cells differentiated into mature neurons as shown by the presence of fast inward currents due to the Na+ channels opening after depolarization with a series of 10 mV voltage steps from −90 mV (the average Resting Membrane Potential (RMP) was equal to −45.9 mV±2.77, FIG. 2A) and their ability to produce action potentials upon stimulation with a depolarizing current, as shown for 7889O (the average RMP was equal to −37.93 mV±6.16, FIG. 2B,C) and 8446B (FIG. 2D).

As further evidence of maturation, 7889O cells exhibited a single action potential following depolarizing current injection at 45 days, while at 55 days they responded with a repetitive AP firing pattern and a rebound action potential after a hyperpolarizing current pulse (FIG. 2C). A similar evolution was observed with 8446B cells, as shown by the presence of multiple action potentials in FIG. 3D. Both inward currents and action potentials were blocked by perfusion with 1 μM tetrodotoxin (TTX, FIG. 2A,B), indicating that the currents were due to activation of Na+ channels. Furthermore, the K+ channel blocker tetraethylammonium (TEA) cancelled the long lasting outward current after the fast inward current, suggesting the presence of K+ channels. At Day 55, 60% of cells were capable of firing trains of action potentials, and 13% had “rebound” action potentials at the end of hyperpolarizing current injections (FIG. 2B,C).

Electrical activity was also assessed by looking at Ca²⁺ activity. Ca²⁺ transients are used by neurons to regulate cellular homeostasis by modulating activity-dependent gene expression, controlling neurotransmitter release, and regulating membrane excitability. Therefore, it was asked whether the neurons displayed normal Ca²⁺ transients that were sensitive to TTX. To measure cytosolic Ca²⁺, neuronally differentiated (45 days) iPSC line 7889O were preloaded with Fluo-4NW and recorded spontaneous Ca²⁺ spikes during a two-minute interval (FIG. 2E). A 500 uM-by-500 uM imaging area was selected in each dish of cells (N=5 dishes), which yielded an average of 34.5 cells per imaging window. Of these cells, 13.2 cells, or approximately 40% rendered measurable Ca²⁺ spikes. A spike frequency of about 0.8 Hz was observed, with an average inter-event interval of 0.045 Hz, approximating what has been observed in both cultured cortical neurons and neurons derived from human iPSCs. After application of 1 uM TTX for 5 minutes, the number of cells that displayed measurable spontaneous Ca²⁺ activity decreased to about 19%, and of those, only a few demonstrated at least one event during the 2-minute imaging time-window. Of the few cells that did show a Ca²⁺ transient, the kinetics were generally slower, again mirroring what has been observed in cultured cortical neurons.

### Example 7

### Aβ Production

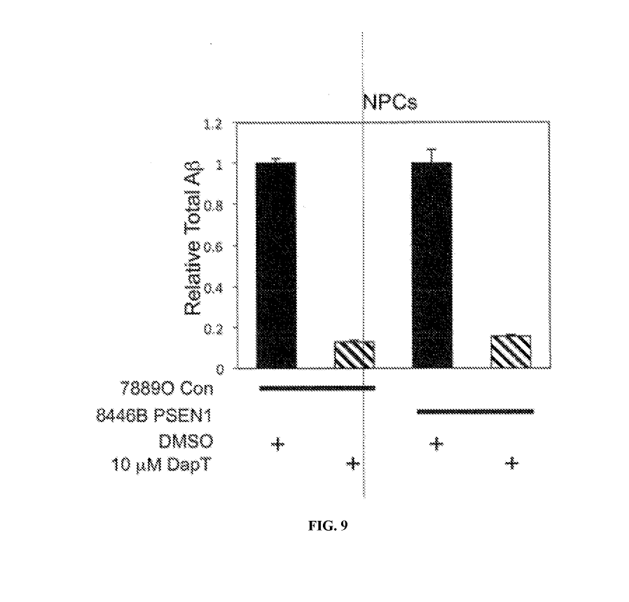

After establishing the authenticity of the functional NPC model, next wished it was investigated how protein processing associated with PS1 function and dysfunction. According to the amyloid hypothesis, oligomerized Aβ peptides are responsible for aberrant synaptic plasticity and cellular toxicity. Fibroblasts from PSEN1 mutant patients have been observed to produce an increased ratio of Aβ42/Aβ40, thus enhancing the relative levels of the more oligomerogenic Aβ42 peptide. In congruence with these earlier studies, an increase in the ratio of Aβ42/Aβ40 secreted by human fibroblasts was also observed via analysis of conditioned media by ELISA (FIG. 3B). Aβ production was looked at in NPCs/early neurons (14-day differentiation) where this parameter has not been previously assessed. The Aβ42/Aβ40 ratio was also increased in conditioned media from PSEN1 NPCs and early neurons as compared to control lines (FIG. 3A). Aβ42/Aβ40 ratio between PSEN1 and control cells appeared to increase in magnitude following neuronal differentiation (FIG. 3B), similar to what has been reported for transdifferentiated PSEN1 neurons. The amount of total A13 produced from control and PSEN1 fibroblasts or NPCs were statistically equivalent (FIG. 3C), indicating that APP processing is altered in PSEN1 cells in terms of quality rather than quantity. Many mutant PS1 proteins have been shown to be hypomorphic; i.e., the total number of moles of both Aβ species generated per mole of APP catabolized. Similar to what has been found in numerous studies in other cell types, a γ-secretase inhibitor (10 μM DAPT) was sufficient to block Aβ production in both control and PS1 cells (FIG. 9).

### Example 8

### Gene Expression Profiling (GEP) Comparison for Control Versus PS1 NPCs

Having established that the molecular pathology of FAD could be successfully modeled in the PSEN1 NPCs, their gene expression was further defined via global gene expression studies. The purpose of this approach was threefold: 1) Further characterization of PSEN1 NPCs, including additional analysis of their neurogenic potential; 2) Identification of molecules that might have a developmental and/or amyloid-independent role in the pathogenesis of FAD; 3) Attempt to find molecules that might also be misregulated in late-onset AD. Thus, GEP experiments were performed on both undifferentiated and 14-day neuronally differentiated control and PSEN1 NPCs using the Illumina HumanHT-12-14 BeadChip platform. The results were analyzed using Genome Studio software, and genes were considered differentially expressed if DiffScores were greater than 13 or less than −13 (p=0.05).

For undifferentiated iPSCs, six lines were used: 7889O, 11C, 6842A, 8446B, 7671C, and 7768C (representing all six patients in the core set; 1 sample per line). There was only one differentially expressed gene between 3 control and 3 PSEN1 lines, NLRP2. However, after neuronal differentiation into NPCs there was a significant increase in gene expression differences. RNA from all 8 core lines was amplified and run in triplicate. 22 of 24 samples were independent biological replicates, while 2 were technical replicates. Control and PS1 cells did not segregate by genotype (FIG. 4A) and when 4 control lines were compared to 4 PSEN1 lines, the majority of genes have overall similar expression as shown by scatter plot (FIG. 4B, correlation coefficient 0.94). Importantly, FAD1 and FAD4 family members also did not segregate by family. This suggests that related controls might not be more beneficial than unrelated controls, although sibling or parental controls might prove more advantageous than the more distant relationships used in this study.

Despite the overall similarity in expression between control and PSEN1 cells, and the lack of clustering by genotype, utilizing the DiffScore parameters described above, there were 206 upregulated genes and 142 downregulated genes in PSEN1 cells relative to control NPC cultures (FIG. 4C). DAVID Functional Annotation was also used on each list to identify relevant gene ontology (GO) terms (Table 2). Using a threshold minimum of 10 genes per GO term, 9 GO terms associated with upregulated genes and 20 GO terms associated with downregulated genes were identified. While some of these terms have overlapping functions, some striking examples include an increase in genes associated with inhibition of gene transcription and a downregulation of apoptosis-related genes (with a notable exception, BIK). Importantly, GO categories associated with neuronal function did not appear at this threshold of analysis. Utilizing the DAVID Functional Clustering Tool with parameters allowing smaller sets of genes, several neuronal GO term categories with 4-5 overlapping genes were detected: GFRA3, ISL1, DLX1, SEMA3B, and ERBB3. Thus, while this supports a subtle increase in neurogenic potential for PSEN1 NPCs, which would be consistent with the observation of a small increase in CD56+ surface expression (FIG. 2C), the overall lack of substantial neuronal GO categories suggests that gene expression differences between PSEN1 and control NPCs are not skewed by the minority of early-born neurons present at day 14 of differentiation.

TABLE 2
Related to FIG. 4: GO Terms associated with differentially regulated
genes as determined by DAVID Functional Annotation.

| | # | |
|---|---|---|
| | Genes | pVALUE |
| ASSOCIATED GO TERMS FOR | | |
| UPREGULATED GENES IN PSEN1 NPCs | | |
| GO:0044421~extracellular region part | 19 | 0.008 |
| GO:0005576~extracellular region | 31 | 0.017 |
| GO:0016481~negative regulation of transcription | 11 | 0.018 |
| GO:0010629~negative regulation of gene expression | 11 | 0.032 |
| GO:0006357~regulation of transcription from RNA | 14 | 0.033 |
| polymerase II promoter | | |
| GO:0045934~negative regulation of nucleobase, | 11 | 0.035 |
| nucleoside, nucleotide and nucleic acid metabolic | | |
| process | | |
| GO:0000166~nucleotide binding | 32 | 0.038 |
| GO:0051172~negative regulation of nitrogen | 11 | 0.038 |
| compound metabolic process | | |
| GO:0043565~sequence-specific DNA binding | 12 | 0.043 |
| ASSOCIATED GO TERMS FOR | | |
| DOWNREGULATED GENES IN PSEN1 NPCs | | |
| GO:0051270~regulation of cell motion | 10 | 7.60E−06 |
| GO:0010033~response to organic substance | 17 | 3.34E−05 |
| GO:0043549~regulation of kinase activity | 12 | 3.61E−05 |
| GO:0051338~regulation of transferase activity | 12 | 5.24E−05 |
| GO:0042325~regulation of phosphorylation | 13 | 8.91E−05 |
| GO:0009719~response to endogenous stimulus | 12 | 1.12E−04 |
| GO:0051174~regulation of phosphorus metabolic | 13 | 1.30E−04 |
| process | | |
| GO:0019220~regulation of phosphate metabolic | 13 | 1.30E−04 |
| process | | |
| GO:0045859~regulation of protein kinase activity | 11 | 1.38E−04 |
| GO:0009725~response to hormone stimulus | 11 | 2.28E−04 |
| GO:0042981~regulation of apoptosis | 16 | 4.06E−04 |
| GO:0043067~regulation of programmed cell death | 16 | 4.51E−04 |
| GO:0010941~regulation of cell death | 16 | 4.69E−04 |
| GO:0042127~regulation of cell proliferation | 14 | 0.003 |
| GO:0005576~extracellular region | 24 | 0.020 |
| GO:0031328~positive regulation of cellular | 11 | 0.020 |
| biosynthetic process | | |
| GO:0009891~positive regulation of biosynthetic | 11 | 0.022 |
| process | | |
| GO:0006915~apoptosis | 10 | 0.024 |
| GO:0012501~programmed cell death | 10 | 0.026 |
| GO:0005886~plasma membrane | 38 | 0.031 |

### Example 9

### gPCR Validation of PSEN1 NPC Differentially-Regulated Genes

It was postulated that most positively and negatively regulated genes in the GEP experiments would be the most potentially relevant to AD. There were 34 upregulated genes (>3-fold increase, Diff Score>13) and 6 downregulated genes (>3-fold elevation in controls, Diff Score<−13) that were attractive candidates for further validation. The upregulated list was further pruned to 23 genes by eliminating 11 genes that were not expressed in control NPCs at statistically detectable levels. This reduced the potential of a rare minority cell type skewing the data.

The remaining 29 genes were analyzed in two independent differentiation experiments utilizing the 8 core cell lines in biological triplicates for each experiment (Table 3). The average expression of each of the four controls lines (n=8, 2 experiments combined) was compared against the average expression of the four PSEN1 lines (n=8, 2 experiments combined). Ten upregulated genes (ASB9, BIK, C7orfl6, NDP, NLRP2, PLP1, SLC45A2, TBX2, TUBB4, ZNF300) and four downregulated genes (ADM2, FLJ35024, MT2A, PTGS2) were validated by this method (Student's t-Test p<0.05). Four additional genes showed an upregulation trend in PSEN1 NPCs (ABCC2, ECEL1, EGFL8, FSTL5, SMOC1). Then the expression of three targets was looked st in more detail: NLRP2, ASB9 and NDP.

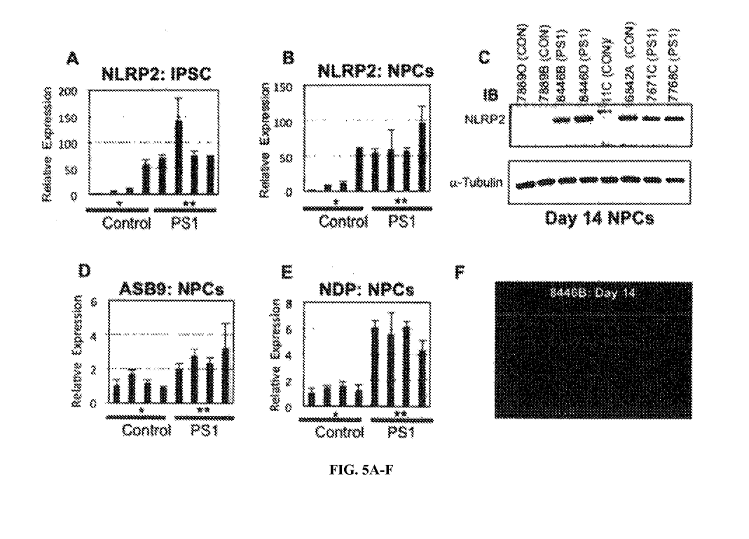

TABLE 3
Related to FIG. 5: qPCR Validation of Gene Expression Profile hits.

| | | | | | | | | | Con | PS1 | | |
|---|---|---|---|---|---|---|---|---|---|---|---|---|
| | 7889O | 7889B | 11C | 6842A | 8446B | 8446D | 7671C | 7768C | Avg | Avg | Ratio | t-Test |
| Higher | | | | | | | | | | | | |
| PS1 | | | | | | | | | | | | |
| ASB9 | 1 | 2.3 | 1.9 | 2.4 | 4.2 | 4.6 | 3.4 | 5.8 | 1.91 | 4.50 | 2.37 | 0.01 |
| BIK | 1 | 1.43 | 1.55 | 1.37 | 2.97 | 4.54 | 7.97 | 0.87 | 1.31 | 4.09 | 3.13 | 0.04 |
| C7orf16 | 1 | 4.72 | 5.73 | 4.70 | 18.48 | 18.44 | 23.12 | 2.52 | 4.04 | 15.64 | 3.88 | 0.03 |
| NDP | 1 | 1.42 | 3.47 | 1.74 | 4.01 | 7.09 | 14.01 | 8.96 | 1.91 | 8.52 | 4.46 | 0.01 |
| NLRP2 | 1 | 4.8 | 6.3 | 42.5 | 48.7 | 53.8 | 43.9 | 70.2 | 20.99 | 54.16 | 2.58 | 0.001 |
| PLP1 | 1 | 1.45 | 2.69 | 3.04 | 4.46 | 9.42 | 13.73 | 8.13 | 2.04 | 8.93 | 4.37 | 0.02 |
| SLC45A2 | 1 | 2.14 | 2.54 | 2.94 | 10.45 | 11.26 | 14.65 | 2.77 | 2.16 | 9.78 | 4.54 | 0.01 |
| TBX2 | 1 | 2.90 | 2.79 | 1.90 | 12.29 | 7.11 | 7.80 | 2.86 | 2.15 | 7.51 | 3.50 | 0.03 |
| TUBB4 | 1 | 1.92 | 2.66 | 2.37 | 8.20 | 14.71 | 19.08 | 1.32 | 1.99 | 10.83 | 5.45 | 0.02 |
| ZNF300 | 1 | 1.32 | 0.74 | 2.65 | 3.04 | 4.84 | 2.72 | 3.91 | 1.42 | 3.63 | 2.55 | 0.02 |
| Lower PS1 | | | | | | | | | | | | |
| ADM2 | 1 | 0.23 | 0.86 | 0.49 | 0.21 | 0.53 | 0.16 | 0.28 | 0.64 | 0.29 | 0.46 | 0.03 |
| FLJ35024 | 1 | 0.38 | 2.06 | 0.16 | 0.06 | 0.08 | 0.13 | 0.48 | 0.90 | 0.18 | 0.20 | 0.04 |
| MT2A | 1 | 0.50 | 0.91 | 0.39 | 0.64 | 0.37 | 0.13 | 0.28 | 0.70 | 0.35 | 0.50 | 0.05 |
| PTGS2 | 1 | 0.91 | 1.89 | 0.22 | 0.41 | 0.30 | 0.16 | 0.26 | 1.00 | 0.28 | 0.28 | 0.02 |
| Trend PS1 | | | | | | | | | | | | |
| ABCC2 | 1 | 8.66 | 6.28 | 6.24 | 24.52 | 31.75 | 71.36 | 1.90 | 8.78 | 34.24 | 3.90 | 0.07 |
| ECEL1 | 1 | 4.93 | 0.94 | 4.58 | 23.13 | 23.43 | 24.98 | 1.41 | 3.14 | 18.24 | 5.81 | 0.07 |
| EGFL8 | 1 | 1.14 | 1.44 | 1.26 | 2.26 | 1.96 | 2.56 | 0.95 | 1.21 | 1.93 | 1.60 | 0.06 |
| FSTL5 | 1 | 4.01 | 3.82 | 2.89 | 5.06 | 6.08 | 14.13 | 3.04 | 2.93 | 7.08 | 2.42 | 0.06 |
| SMOC1 | 1 | 1.08 | 1.70 | 1.07 | 3.80 | 6.05 | 14.49 | 0.78 | 1.21 | 6.28 | 5.19 | 0.08 |

Relative expression values for each cell line reflect the averages of 2 independent experiments with 3 biological replicates per experiment. Student's t-Test reflects differences between control and PSEN1 NPCs and was calculated using data points from two independent experiments rather than the average listed.

### Example 10

### NLRP2, ASB9, and NDP Expression in NPCs

NLRP2 (NALP2) was the only gene differentially regulated in undifferentiated iPSC GEPs. It is also one of the 10 PSEN1 upregulated genes found in the NPC analysis. NLRP2 is a component of the inflammasome, a protein complex that activates pro-inflammatory caspases such as caspase-1. As inflammation has been argued to play a critical role in AD, it is intriguing that a major modulator of inflammatory signaling might be different in FAD patients from birth. This becomes particularly interesting in the recent report where crossing of an FAD mouse model with either NLRP3 or caspase-1 null animals caused decreased Aβ accumulation and significant attenuation of synaptic and memory deficits.

It was confirmed that NLRP2 expression was upregulated in undifferentiated PSEN1 cells. As shown in FIG. 5A, NLRP2 was expressed at much higher levels in the 4 PSEN1 iPSC lines as well as control iPSC line 6842A (average 15-fold higher for those 5 lines compared to the other 3 control lines). Similar results were confirmed in PSEN1 NPCs and 6842A at the mRNA and protein levels (FIG. 5B,C). Interestingly, NLRP2 is located on chromosome 19 at the breakpoint of the balanced translocation (q13.42) present in iPSC line 6842A.

ASB9 is a broadly expressed E3-ligase that targets creatine kinase B (CKB) and ubiquitous mitochondrial creatine kinase for degradation, and stable overexpression of ASB9 reduces mitochondrial membrane potential and affects mitochondrial morphology. CK activity has been shown to be lower in AD brains, and creatine has shown to be neuroprotective and is in clinical trials for multiple neurodegenerative diseases. The upregulation of ASB9 over 2.4-fold in PS1 NPCs was confirmed (FIG. 5D). ASB9 protein was not detected in the cells by immunostaining or Western blot. It is perhaps not surprising that it might be expressed at low levels due to its deleterious effects on mitochondrial function.

Mutations in Norrie Disease Pseudoglioma (NDP, protein called Norrin) are responsible for Norrie disease, an X-linked recessive disorder, as well as several other rare eye disorders. The primary manifestation of Norrie Disease is blindness and, in many cases, progressive hearing loss. In addition, in 30% or more of patients, mental retardation is also present, suggesting an important CNS role for NDP. Thus, NDP became an interesting target for further study, particularly in light of the hypothesis that FAD might have a developmental component. That NDP is in fact upregulated in PSEN1 NPCs was confirmed by qPCR (FIG. 5E). The aggregated data from three independent experiments indicated that NDP was upregulated 5.5 fold in PSEN1 NPCs (p=0.0002, Student's t-Test).

### Example 11

### Examination of PSEN1 NPC Differentially-Regulated Genes in Late Onset AD Brains

Next, it was decided to determine if the differentially-expressed genes in PSEN1 NPCs could be reflected in brains from AD patients. Only Late-onset AD brains were analyzed as brain tissue from FAD individuals was not obtained. In addition, while the iPSC-derived NPCs may more closely resemble embryonic NPCs or adult NPCs rather than mature neurons, it was hypothesized that some of the NPC hits might be altered in late-onset AD neuronal populations.

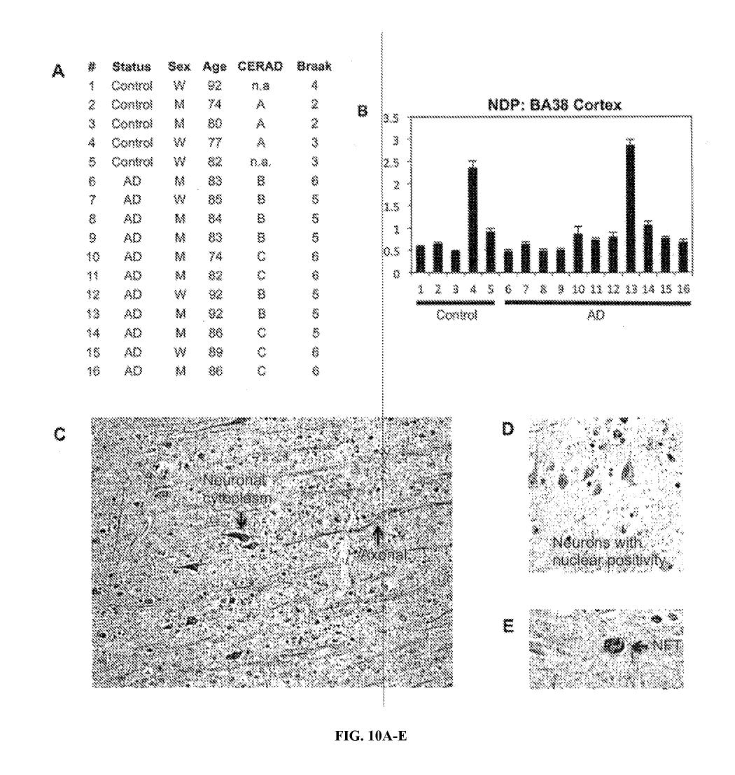

The first approach was to analyze NLRP2, ASB9, and NDP expression in AD brains by qPCR. mRNA isolated from Broadmann Area 38 (BA38, temporal pole) of 11 AD and 5 control patients was converted to cDNA and analyzed by qPCR for NLRP2, ASB9 and NDP. The temporal lobe (including the temporal pole) is vulnerable in Alzheimer's disease, particularly in intermediate stages of the disease. Interestingly, NLRP2 expression was found to be statistically reduced in BA38 of AD patients, the opposite of the NPC results (38% of levels of controls, p=0.005, Student's t-Test, FIG. 6A). ASB9 was elevated in BA38 of some AD individuals, but was only a trend when looking at all 11 AD patients compared to controls (FIG. 6B). NDP expression was similar between control and AD patients by qPCR, but intriguingly was present in some neurofibrillary tangles in AD forebrains (FIG. 10).

To further confirm the authenticity of hits, publically available GEPs from laser capture microdissected (LCM) cortical neurons from control subjects or from patients with moderate or severe AD pathology were analyzed. Some individuals displayed intermediate pathology individuals but without sufficient clinical criteria to be labeled as having AD; these cases were designated “non-demented individuals demonstrating AD pathology” (NDAD). The list of 14 differentially expressed genes was used to interrogate GEPs of hippocampus, entorhinal cortex (EC), and the middle temporal gyrus (MTG) from NDAD individuals and hippocampus from AD patients. Gene expression from these regions had already been compared to brain regions from age-matched control patients, and thus one could determine whether the genes misregulated in mutant PSEN1 NPCs were similarly misregulated in early stages of AD pathology in vulnerable brain areas.

Five target genes had statistically altered expression in AD/AD pathology brains (FIG. 6C). C7orfl6 (PPP1R17) and ZNF300, which are elevated in PSEN1 NPCs, were also found to be more highly expressed in NDAD hippocampi and NDAD MTG respectively. One caveat is that C7orfl6 was expressed at very low levels. MT2A, which has reduced expression in PSEN1 NPCs, was also expressed at significantly lower levels in NDAD hippocampi and EC. Interestingly, two other metallothionein proteins, MT1A and MT1F, were also significantly reduced in the GEP of PSEN1 NPCs. Some studies report that MTs may attenuate Aβ toxicity. On the other hand, NDP, which is elevated in PSEN1 NPCs, is significantly lower in both NDAD and AD hippocampi. The situation with TUBB4 (elevated in PSEN1 NPCs) is more complex, as it is lower in NDAD EC, but increased as a trend in NDAD MTG. There was a trend toward upregulation of ASB9, although this did not reach statistical significance. While NLRP2 itself was not differentially regulated in the brain regions analyzed, its homologue NLRP1 was downregulated in MTG of brains designated NDAD (37%, p=0.00005). Overall, the data support the hypothesis that at least some of the hits of genes differentially regulated in FAD NPCs are also differentially regulated in AD brains.

### Example 12

### Recombinant Norrin Promotes Primary Neurosphere Formation in Adult SVZ Progenitors

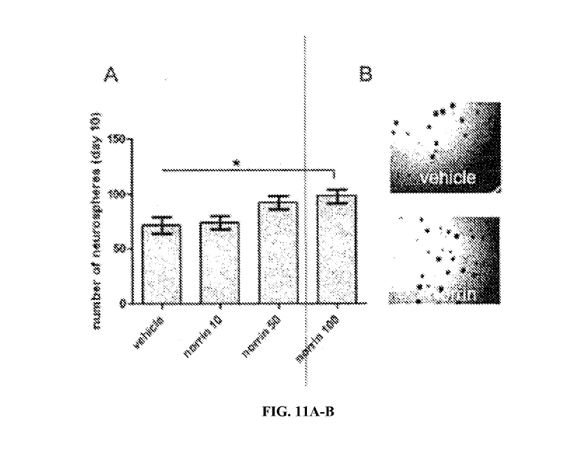

Norrin is a secreted molecule that can activate canonical Wnt signaling via Frizzled-4 and has been shown to also inhibit TGFβ signaling. While Wnt signaling has multiple roles during development, it has been shown to be critical for adult neurogenesis, and has been shown to be impaired in neurospheres isolated from recently-deceased AD patients. Decreased NDP expression in the hippocampus of both late-onset AD brains and NDAD brains was found (FIG. 6), suggesting a role for NDP in adult neurogenesis, therefore tested recombinant norrin was tested on primary murine adult subventricular zone (SVZ) progenitors as well as human iPSC-derived NPCs for effects of on proliferation. iPSC-derived NPCs (d14) did not show differences in proliferation in response to norrin (ki67 staining, data not shown), in line with the observation PS1 cells do not show greater proliferation at this time point of differentiation despite greater endogenous levels of NDP expression (ki67 staining, FIG. 1G). However, adult SVZ progenitors from eight-week old mice did show increased proliferation in response to recombinant norrin, as measured by primary neurospehere formation, even in the presence of the mitogens EGF and FGF (p=0.04; FIG. 11). This supports a potential pro-neurogenic role for NDP in adult neurogenesis.

### Example 13

### Methods

Cell Lines:

Fibroblast 11 and 11C have been previously published (Bock et al., Cell 144:439 (2011)). All other fibroblasts were obtained from the Coriell Institute (Camden, N.J.), and were reprogrammed to iPSCs in this study.

Molecular Biology:

Genomic DNA was prepared using the DNA Mini Kit, and RNA with the RNeasy Mini Kit, as per the manufacturer's instructions (Qiagen). qPCR was carried out on a Stratagene MX3000P QPCR machine (Agilent technologies) utilizing 40 cycles. cRNA for was amplified using the Illumina TotalPrep RNA Amplification Kit (Ambion) and ran on an Illumina HT_12_v4 BeadChip Array (Ilumina), as per the manufacturer's instructions. Oligos are found in FIG. 11.

Protein Analysis:

Immunostaining was performed as has been described before (Sproul et al., Cell Res 19:950 (2009)). Hoescht 33342 (Sigma) were used to visualize DNA. The following antibodies were used: OCT4 (Stemgent), SSEA4, Nanog (R&D Systems), Tra-160, Ki67, MAP2, Nestin, NeuN (Millipore), Tuj1 (Covance), NLRP2 (Santa Cruz), and NDP (Abnova). Quantification of immunostaining was done on the Celigo 200-BFFL Machine (Brooks Automation).

Aβ Assays:

Fibroblasts were split at 100,000 cells/6 well-well, and allowed to condition for 3 days before collection of conditioned media for analysis. Neuronal cultures were also conditioned for 3 days prior to collection of conditioned media at day 14. To quantify Aβ levels, human/rat Aβ 1-40 and 1-42 ELISA kits (Wako) were used according to the manufacturer's instructions. Duplicate assays were averaged for each biological replicate. There were 3 biological replicates for each line in each experiment (three independent-experiments per cell type). Assays were performed blindly.

Human Brain Tissue Analysis:

De-identified fresh frozen human autopsy brain tissue was obtained from the New York Brain Bank at Columbia University Medical Center (New York, N.Y.). Neuropathological examination was per standardized protocols (Vonsattel et al., Cell Tissue Bank 9:247 (2008); Vonsattel et al., Acta Neuropathologica 115:509 (2008)).

Retroviral Reprogramming:

Fibroblasts were plated at 30,000-50,000 cells in a single well of a 6-well plate or multiple wells of a 12-well-plate, which was infected the 6-18 hours later with four retroviruses prepared for Oct4, KLF4, Sox2, and c-Myc by the Harvard Gene Therapy Core. Infected fibroblasts were split 7-14 days post-infection onto MEFs (γ-irradiated mouse embryonic fibroblasts) and concomitantly treated with three chemical compounds to enhance reprogramming: SB431512 (2 μm), Thiazovivan (0.5 μM), and PD0325901 (0.5 μM)(Lin et al., 2009).

Cell Culture:

Cell culture reagents were from Invitrogen unless otherwise stated, and all media contained penicillin-streptomycin (100U/mL-0.1 mg/mL). Human fibroblasts were maintained on TC plates coated with 0.1% gelatin and grown in FM10 Media (DMEM/10% FBS/Glutamax (2 mM)/2-Mercaptoethanol (0.1 mM). Cells were initially quarantined until they were tested for the absence of mycoplasma. Undifferentiated iPSCs were kept on irradiated MEFs (Globalstem) plated on TC plates with 0.1% gelatin, and grown in HUESM (20% KSR/KO-DMEM/Non-essential amino acids (0.1 mM)/Glutamax (2 mM) 2-Mercaptoethanol (0.1 mM)/bFGF (10 ng/ml)). Monolayer neuronal differentiation was carried out in custom mTesR1 (minus 5 growth factors) and Neurobasal media supplemented with B-27 minus retinoic acid. PD0325901, SB431542, Thiazovivin, and LDN-193189 were from Stemgent. Matrigel, natural mouse laminin and polyornithine were also from Invitrogen. Karyotyping and fingerprinting of cell lines was done by Cell Line Genetics (Madison, Wis.).

Monolayer Neuronal Differentiation:

Briefly, iPSC colonies grown on MEFs were cleaned to remove differentiated cells and pre-plated on gelatin-coated plates to remove MEFs. The resulting SNF was spun and resuspended in mTesR1 media containing 10 1 AM ROCK inhibitor (Y-27632, Stemgent), plated at a density of 200,000 cells per 6 well-well on polyornithine (100 n/mL)/laminin (3 μg/mL), and allowed to recover for 3 days to allow near confluency. Cells were neuronally differentiated with dual-smad inhibition from days 0-9 in custom TesR1 (5× supplement, 1/100 Pen-Strep) using 10 μM SB431542 and 250 nM LDN193189. Cell were split with acutase on Day 9, and plated in a similar density and substrate as the initial plating. Media was changed from custom mTESR1 in Neurobasal+ B27 supplement (no retinoic acid) in a stepwise fashion, and cells were fed every 2 to 3 days until analyzed.

Flow Cytometry for CD56:

Cells were enzymatically harvested with Accutase above to obtain a single cell suspension prior to resuspension in 100 μl of a sterile iPSC staining buffer [DPBS containing 0.5% bovine serum albumin fraction V (BSA; Invitrogen), 100 U/ml penicillin/streptomycin (Invitrogen), 2 mM EDTA (Invitrogen), and 20 mM glucose (Sigma)]. Fluorescence-conjugated CD56 (1 μl anti-CD56, catalog #s: 560360, BD Biosciences) were added to cells that had been filtered through a 35 urn cell strainer (BD Biosciences). Cells were incubated at room temperature (RT) for 20 minutes shielded from light. The stained cells were washed once with iPSC staining buffer and were analyzed immediately on a 5 laser BD Biosciences ARIA-IIu™ SOU Cell Sorter configured with a 100 μm ceramic nozzle and operating at 20 psi sheath fluid pressure.

Electrophysiology:

Neurons were cultured on polyornithine and laminin coated coverslips and recorded using whole cell patch between day 35 and 55. The recording bath solution consisted of (in mM): NaCl (119), KCl (5), HEPES (20), Glucose (30), MgCl 2 (2), CaCl² (2), glycine (0.001), and picrotoxin (0.1). Glass electrodes with a resistance between 5.5-7 MO, were filled with an intracellular solution containing (in mM): K-Gluconate (130), KCl (10), HEPES (5), CaCl² (0.06), MgCl₂ (5), EGTA (0.6), ATP (2), GTP (0.2), Phosphocreatine (20) and Leupeptine (0.2) and 50 U/ml creatine-phosphokinase. Recordings were made with a Warner amplifier (model PC-501A) and filtered at 1 kHz. Signals were sampled at 10 kHz with 16-bit resolution with a Digidata 1440A (Axon Instrument), and analyzed with Matlab 8.0 (MathWorks). To evoke action-potentials, current clamp steps were given (0 pA for 100 ms, steps from −60 pA to +120 pA, 20 pA each, for 1 s). Action potentials were subsequently blocked with perfusion of 1 pM TTX. Na²⁺ and K⁺— currents were obtained in voltage clamp mode (holding potential: −70 mV for 100 ms, steps from −90 mV to +20 mV, 10 mV each, for 1 s). Sodium and potassium currents were blocked through 1 pM TTX and 10 mM TEA, respectively.

Calcium Studies:

iPSC line 78890 neuronal cultures that had been neuronally differentiated for 45 days were pretreated with an HBSS loading buffer containing 20 mM HEPES, 2.5 mM probenecid, and Fluo-4NW (Invitrogen) for 30 min at 37° C. and then at room temperature for an additional 30 min before imaging, following the manufacturer's instructions. Cells were then placed in an imaging chamber and imaged on a Zeiss LSM 510 inverted confocal microscope using a 20× objective (0.8 n.a.). Time-lapse images were taken every 1.5 seconds for 3 minutes using 512×512 pixel resolution and cells were excited using the 488 nm laser line. 4 uM×4 uM ROIs were drawn over each cell soma and change in fluorescence intensity over time was calculated within the imaging software. After background subtraction, Ca2+ signals were then analyzed by hand and with a custom written routine in Matlab (Mathworks, Natick, Mass.) based on the “peakfinder” and “relevantpeaks” functions in Matlab. Observers were blind to each condition.

Recombinant Norrin Treatment of SVZ Progenitors:

Adult FVB/N mice were killed by cervical dislocation (n=3 per preparation) and their brains removed and placed into Pipes buffer (20 mM Pipes, 25 mM glucose, 0.12M NaCl, 0.5 mM KCl [ph 7.4]). The lateral walls of the lateral ventricles were dissected, collected in Pipes buffer and incubated in a mixture containing 1.2 mL of activated papain and 8.8 ml of Pipes buffer. Papain was activated 20 min in advance by placing 3 mg of papain (Worthington Biochemical) in 1 ml of 1.1 mM EDTA, 5.5 mM cysteine HCl. The pieces of tissue were incubated in papain solution at 37° C. on a rocking platform for 10 min and collected by centrifugation at 1300 rpm for 5 min. The cellular pellet was resuspended in 1 ml of DMEM-F12 (GIBCO) containing 0.7 mg of ovomucoid inhibitor and 1 mg of DNase. The cells were dissociated and neural precursor cells were separated from differentiated cells, myelin and extracellular matrix using density gradient separation by centrifugation step with 22% Percoll. Pelleted cells were separated from supernatant, washed and resuspended in 1 ml neurosphere proliferation media (DMEM-F12 (GIBCO) supplemented with N2 (GIBCO), 2 mM glutamine, 0.6% (w/v glucose), insulin-transferrin-selenium (GIBCO), and 15 mM HEPES, B27, penicillin/streptomycin). The cells were counted and plated in 96well-low attachment plate (Corning) (1.5×10⁵/mL) in the same medium containing 10 ng/mL of EGF receptor grade (Millipore) and 10 ng/mL human bFGF (Invitrogen). Two days after isolation neurospheres were grown in the presence of different concentrations (10, 50 and 100 ng/mL) of the recombinant Norrin protein (R&D systems), media, or vehicle control (Norrin was resuspended in 4 mM HCL/0.1% BSA). The numbers of neurospheres were counted for six 40× fields/well after 10 days in vitro. Each experiment was performed in triplicate for each condition and a total of three independent experiments were included.

Analysis of Laser Captured Microdissected (LCM) Neuron GEP Data:

Analysis was performed on a GEP dataset (GSE5281) downloaded from the Gene Expression Omnibus (GEO) database. The CEL files were downloaded and analyzed using BRB ArrayTools (available at linus.nci.nih.dov/BRB-ArrayTools.html). The CEL files were normalized and filtered prior to comparing gene expression levels. First, the downloaded CEL files were normalized using the MASS algorithm. Next, probesets that had the lowest variance across the samples were filtered out. The significance of the variance was calculated for each probeset by comparing the variance of their log-expression values to the median of all the variances. Statistical significance was measured using the corrected FDR value and probesets that had a variance of less than the FDR-corrected p-value<0.1 were filtered out. Out of 54,000 total probesets on the array, 17,224 probesets passed this filter. Comparison of gene expression levels was performed on the normalized and filtered dataset and significance was measured with the FDR-corrected p-value. The following are the number of individuals analyzed per brain region: Hippocampus: Control 13, NDAD 6, AD 10; Entorhinal Cortex: Control 13, NDAD 6; Middle Temporal Gyrus: Control 12, NDAD 6, AD 16

Oligos:

QPCR Primers
Oct 4 (endogenous)

(SEQ ID NO: 1)

F: CCCCAGGGCCCCATTTTGGTACC

(SEQ ID NO: 2)

R: GGCACAAACTCCAGGTTTTC
Sox2 (endogenous)

(SEQ ID NO: 3)

F: ACACTGCCCCTCTCACACAT

(SEQ ID NO: 4)

R: GGGTTTTCTCCATGCTGTTTCT
Klf4 (endogenous)

(SEQ ID NO: 5)

F: ACCCACACAGGTGAGAAACCTT

(SEQ ID NO: 6)

R: GTTGGGAACTTGACCATGATTG
c-Myc (endogenous)

(SEQ ID NO: 7)

F: AGCAGAGGAGCAAAAGCTCATT

(SEQ ID NO: 8)

R: CCAAAGTCCAATTTGAGGCAGT
Oct4 (transgene)

(SEQ ID NO: 9)

F: CCCCAGGGCCCCATTTTGGTACC

(SEQ ID NO: 10)

R: AACCTACAGGTGGGGTCTTTCA
Sox2 (transgene)

(SEQ ID NO: 11)

F: ACACTGCCCCTCTCACACAT

(SEQ ID NO: 12)

R: AACCTACAGGTGGGGTCTTTCA
Klf4 (transgene)

(SEQ ID NO: 13)

F: GACCACCTCGCCTTACACAT

(SEQ ID NO: 14)

R: AACCTACAGGTGGGGTCTTTCA
c-Myc (transgene)

(SEQ ID NO: 15)

F: AGCAGAGGAGCAAAAGCTCATT

(SEQ ID NO: 16)

R: AACCTACAGGTGGGGTCTTTCA
B2M

(SEQ ID NO: 17)

F: TAGCTGTGCTCGGGCTACT

(SEQ ID NO: 18)

R: TCTCTGCTGGATGACGCG
GAPDH

(SEQ ID NO: 19)

F: ACATCGCTCAGACACCATG

(SEQ ID NO: 20)

R: TGTAGTTGAGGTCAATGAAGGG
ABCC2

(SEQ ID NO: 21)

F: AGGAGATTTGGCTGAGATTGG

(SEQ ID NO: 22)

R: TTTCCTACATGAGCATCCACTG
ADM2

(SEQ ID NO: 23)

F: GACCCGTCAAACCCAGG

(SEQ ID NO: 24)

R: GAGGCTGACCCATAACAGG
ASB9

(SEQ ID NO: 25)

F: ACGGCAGATCATGTTTCCC

(SEQ ID NO: 26)

R: CACAAGCATTAAACAGTGGAGTG
BIK

(SEQ ID NO: 27)

F: TCTTTGGAATGCATGGAGGG

(SEQ ID NO: 28)

R: GTAGATGAAAGCCAGACCCAG
C7orf16

(SEQ ID NO: 29)

F: TCTTCATAACACTGACCTGGAAC

(SEQ ID NO: 30)

R: ACCATCCTTTTCGTCATCTTCC
ECEL1

(SEQ ID NO: 31)

F: CCCTCTTTGTACATGAGCACT

(SEQ ID NO: 32)

R: ACTTGATGTCTTCCACTAGCTG
EGFL8

(SEQ ID NO: 33)

F: AACCAGTGCCAGCATACTC

(SEQ ID NO: 34)

R: CTCGTGAATCTCCTGCTTCAG
FLJ35024

(SEQ ID NO: 35)

F: CAAACAAGAGGTCCCTGGATC

(SEQ ID NO: 36)

R: AGTTGATGCCTGGTCTGAAG
FSTL5

(SEQ ID NO: 37)

F: GATCAGGTCTGGGTGCTAAG

(SEQ ID NO: 38)

R: CCACTCTGTCAAATTGCTTTCC
MT2A

(SEQ ID NO: 39)

F: GGCTCCTGCAAATGCAAA

(SEQ ID NO: 40)

R: CAGCAGCTGCACTTGTCC
NDP

(SEQ ID NO: 41)

F: GCGTCACGCTCCGAGCCTTT

(SEQ ID NO: 42)

R: CTGAGCATCGCAGCCGCAGT
NLRP2:

(SEQ ID NO: 43)

F: CTTGGTGCTTTGGAACTGCGACAT

(SEQ ID NO: 44)

R: TTGCACAGTGGTTTCCTCAAAGCC
PLP1

(SEQ ID NO: 45)

F: CTGGCTGAGGGCTTCTACAC

(SEQ ID NO: 46)

R: CCTAGCCATTTTCCCAAACA
PTGS2:

(SEQ ID NO: 47)

F: ACAGGCTTCCATTGACCAG

(SEQ ID NO: 48)

R: TCACCATAGAGTGCTTCCAAC
SLC45A

(SEQ ID NO: 49)

F: AGACCCTACATCCTCACCC

(SEQ ID NO: 50)

R: AAAGAGAACGACACCTATCATGG
SMOC1

(SEQ ID NO: 51)

F: TGGTTCAGGCCATTAACTCAG

(SEQ ID NO: 52)

R: TCTCCCGCTTGTTAATGTCG
TBX2

(SEQ ID NO: 53)

F: AGTTCCACAAGCTAGGCAC

(SEQ ID NO: 54)

R: ACTTATAGCGGCAATCGTCAG
TUBB4A:

(SEQ ID NO: 55)

F: TGCAACTGGAGAGGATCAAC

(SEQ ID NO: 56)

R: CCGAAAGATCTGACCGAAGG
ZNF300

(SEQ ID NO: 57)

F: CAGTTTCCAAACCAGATGTCATC

(SEQ ID NO: 58)

R: ACTGGGAGTTGTGAAGGTTAC
Southern Blot Probes
Oct4

(SEQ ID NO: 59)

F: GAGAAGGAGAAGCTGGAGCA

(SEQ ID NO: 60)

R: GTGAAGTGAGGGCTCCCATA
Klf4

(SEQ ID NO: 61)

F: ACCTGGCGAGTCTGACATGG

(SEQ ID NO: 62)

R: TCTTCATGTGTAAGGCGAGGTGG

Those of skill in the art should, in light of the present disclosure, appreciate that many changes can be made in the specific embodiments which are disclosed and still obtain a like or similar result without departing from the spirit and scope of the invention. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Accordingly, the invention is limited only by the following claims.

What is claimed is:

1. A method of diagnosing, prognosing, determining progression of Alzheimer's disease (AD), predicting a therapeutic regimen or predicting benefit from therapy in a subject having Alzheimer's disease comprising:
(a) assaying an expression level of one or more targets in a sample from the subject, selected from the group consisting of NLRP2, ASB9, BIK, C7orfl6, ZNF300 and ADM2 and any combination thereof; and
(b) diagnosing, prognosing, determining progression of AD, predicting a therapeutic regimen or predicting benefit from therapy in a subject having AD based on the expression levels of the plurality of targets.

2. The method of claim 1, wherein the plurality of targets further comprises NDP, PLP1, SLC45A2, TBX2, TUBB4, FLJ35024, MT2A, PTGS2, ABCC2, ECEL1, EGFL8, FSTL5, or SMOC1 or any combination thereof.

3. The method of claim 1, wherein ASB9, BIK, C7orfl6, NDP, NLRP2, PLP1, SLC45A2, TBX2, TUBB4 and/or ZNF300 are upregulated.

4. The method of claim 1, wherein ADM2, FLJ35024, MT2A and/or PTGS2 are downregulated.

5. A system for analyzing Alzheimer's disease in a subject, comprising:
(a) a probe set comprising a plurality of target sequences, wherein
(i) the plurality of target sequences hybridizes to more than one target selected from the group consisting of NLRP2, ASB9, BIK, C7orfl6, ZNF300 and ADM2 or a combination thereof;
(b) a plurality of probes that hybridize to the plurality of target sequences;
(c) a sample from a subject having Alzheimer's disease;
(d) a computer model or algorithm for analyzing an expression level and/or expression profile of the targets hybridized to the probes in a sample from a subject having Alzheimer's disease.

6. The system of claim 5, further comprising an electronic memory for capturing and storing an expression profile.

7. The system of claim 5, further comprising a computer-processing device, optionally connected to a computer network.

8. The system of claim 7, further comprising a software module executed by the computer-processing device to analyze an expression profile.

9. The system of claim 7, further comprising a software module executed by the computer-processing device to compare the expression profile to a standard or control.

10. The system of claim 7, further comprising a software module executed by the computer-processing device to determine the expression level of the target.

11. The system of claim 5, further comprising a machine to isolate the target or the probe from the sample.

12. The system of claim 5, further comprising a machine to sequence the target or the probe.

13. The system of claim 5, further comprising a machine to amplify the target or the probe.

14. The system of claim 5, further comprising a label that specifically binds to the target, the probe, or a combination thereof.

15. The system of claim 7, further comprising a software module executed by the computer-processing device to transmit an analysis of the expression profile, a diagnosis and/or a prognosis to the subject or a medical professional treating the subject.

16. The system of claim 5, wherein the target further comprises NDP, PLP1, SLC45A2, TBX2, TUBB4, FLJ35024, MT2A, PTGS2, ABCC2, ECEL1, EGFL8, FSTL5, or SMOC1 or a combination thereof.

17. A method of treating a subject with Alzheimer's disease comprising:
(a) obtaining an expression profile of a plurality of targets from a sample from a subject;
(b) determining the subject has Alzheimer's disease by comparing the expression profile from the patient to an expression profile from a control or standard; and
(c) administering a therapeutic agent or treatment to the subject,
wherein the plurality of targets comprises one or more targets selected from the group consisting of NLRP2, ASB9, BIK, C7orfl6, ZNF300 and ADM2 or a combination thereof.

18. The method of claim 17, wherein the plurality of targets further comprises NDP, PLP1, SLC45A2, TBX2, TUBB4, FLJ35024, MT2A, PTGS2, ABCC2, ECEL1, EGFL8, FSTL5, or SMOC1 or a combination thereof.

19. The method of claim 17, wherein ASB9, BIK, C7orfl6, NDP, NLRP2, PLP1, SLC45A2, TBX2, TUBB4 and/or ZNF300 are upregulated.

20. The method of claim 17, wherein ADM2, FLJ35024, MT2A and/or PTGS2 are downregulated.

21. The method of claim 17, wherein the treatment is an acetylcholinesterase inhibitor or a an NMDA receptor antagonist.

22. The method of claim 17, wherein the treatment is selected from the group consisting of tacrine, rivastigmine, galantamine, donepezil and memantine.

23. A method of identifying an agent for the treatment of Alzheimer's disease comprising:
(a) contacting a cell with a test agent, wherein the expression of a plurality of targets in the cell is consistent with Alzheimer's disease;
(b) determining the expression of the plurality of targets from the cell;
(c) comparing the expression of the plurality of targets from the cell to a control or standard; and
(d) determining that the expression of the plurality of targets is not consistent with Alzheimer's disease,
wherein the plurality of targets comprises one or more targets selected from the group consisting of NLRP2, ASB9, BIK, C7orfl6, ZNF300 and ADM2 or a combination thereof.

24. The method of claim 23, wherein the plurality of targets further comprises NDP, PLP1, SLC45A2, TBX2, TUBB4, FLJ35024, MT2A, PTGS2, ABCC2, ECEL1, EGFL8, FSTL5, or SMOC1 or a combination thereof.

25. The method of claim 23, wherein up regulation of ASB9, BIK, C7orfl6, NDP, NLRP2, PLP1, SLC45A2, TBX2, TUBB4 and/or ZNF300 is consistent with Alzheimer's disease.

26. The method of claim 23, wherein downregulation of ADM2, FLJ35024, MT2A and/or PTGS2 is consistent with Alzheimer's disease.

27. A kit for analyzing Alzheimer's disease, comprising:
(a) a probe set comprising a plurality of target sequences, wherein the plurality of target sequences hybridizes to more than one target selected the group consisting of NLRP2, ASB9, BIK, C7orfl6, ZNF300 and ADM2 or a combination thereof; and
(b) a computer model or algorithm for analyzing an expression level and/or expression profile of the target sequences in a sample or a combination thereof.

28. The kit of claim 27, further comprising a computer model or algorithm for correlating the expression level or expression profile with disease state or outcome.

29. The kit of claim 27, further comprising a computer model or algorithm for designating a treatment modality for the subject.

30. The kit of claim 27, further comprising a computer model or algorithm for normalizing expression level or expression profile of the target sequences.

31. The method of claim 27, wherein the target further comprises NDP, PLP1, SLC45A2, TBX2, TUBB4, FLJ35024, MT2A, PTGS2, ABCC2, ECEL1, EGFL8, FSTL5, or SMOC1 or a combination thereof.

32. A system for analyzing Alzheimer's disease (AD) comprising:
(a) a computer processing device for determining an expression profile for a plurality of targets;
(b) an electronic memory device for capturing and storing an expression profile;
(c) a software module;
(d) a machine to isolate target or the probe from the sample;
(e) a machine to sequence the target or the probe;
(f) a machine to amplify the target or the probe;
(g) a computer model or algorithm for analyzing an expression level and/or expression profile of the target hybridized to the probe in a sample from a subject suffering from AD; and
(h) an output providing the analysis.

33. The system of claim 32, wherein the a plurality of targets comprises wherein one or more targets selected from the group consisting of NLRP2, ASB9, BIK, C7orfl6, ZNF300 and ADM2 or a combination thereof.

34. The system of claim 33, wherein the plurality of targets further comprises NDP, PLP1, SLC45A2, TBX2, TUBB4, FLJ35024, MT2A, PTGS2, ABCC2, ECEL1, EGFL8, FSTL5, or SMOC1 or a combination thereof.

35. The system of claim 32, wherein the software module executed by the computer-processing device analyzes an expression profile.

36. The system of claim 32, wherein the software compare executed by the computer-processing devices the expression profile to a standard or control.

37. The system of claim 32, wherein the software module executed by the computer-processing device determines the expression level of the target.

38. The system of claim 32, wherein the software module executed by the computer-processing device transmits an analysis of the expression profile to the subject or a medical professional treating the subject.

39. The system of claim 32, further comprising a label that specifically binds to the target, the probe, or a combination thereof.
